(19) United States
(12) Patent Application Publication
(10) Pub. No.: US 2023/0080350 A1
(43) Pub. Date: Mar. 16, 2023

(54) Methods and Apparatus for Diagnosis of Progressive Kidney Function Decline Using a Machine Learning Model

(71) Applicants: ICAHN SCHOOL OF MEDICINE AT MOUNT SINAI, New York, NY (US); RENALYTIX AI PLC, Penarth, Cardiff (GB)

(72) Inventors: Girish N. NADKARNI, New York, NY (US); Steven G. COCA, New York, NY (US); Fergus FLEMING, Penarth, Cardiff (GB)

(21) Appl. No.: 17/797,400

(22) PCT Filed: Feb. 12, 2021
(86) PCT No.: PCT/US21/18030

§ 371 Date: Aug. 3, 2022

Related U.S. Application Data
(60) Provisional application No. 63/016,868, filed on Apr. 28, 2020.
(60) Provisional application No. 62/976,767, filed on Feb. 14, 2020.
(60) Provisional application No. 62/976,761, filed on Feb. 14, 2020.

(51) Int. Cl. G16H 50/20; G16B 20/00; G16B 40/20

(52) U.S. Cl. CPC G16H 50/20; G16B 20/00; G16B 40/20

(57) ABSTRACT

In some embodiments, a non-transitory processor-readable medium can store code to be executed by a processor of a first compute device. The code can include code to cause the processor to receive, from a second compute device remote from the first compute device, a trained machine learning model. The code can include code to cause the processor to receive biomarker data and HSD of a diabetic human subject. The biomarker data can indicate a level of at least one of the following biomarkers: sTNFR-1, sTNFR-2, KIM-1, and ratios to one another of any of the preceding. The HSD can include a metabolic factor, a health-related factor, or a demographic-related factor. The code can include code to cause the processor to execute the trained machine learning model to generate an indication of whether the diabetic human subject will experience a progressive decline in kidney function over a period of time.

### CROSS REFERENCE TO RELATED APPLICATION

This application claims priority to and the benefit of U.S. Patent Application Ser. No. 62/976,767, filed Feb. 14, 2020 and titled “MACHINE LEARNING FOR RAPID KIDNEY FUNCTION DECLINE” U.S. Patent Application Ser. No. 62/976,761, filed Feb. 14, 2020 and titled “DERIVATION AND VALIDATION OF A MACHINE LEARNING RISK SCORE USING BIOMARKER AND ELECTRONIC PATIENT DATA TO PREDICT RAPID PROGRESSION OF DIABETIC KIDNEY DISEASE;” and U.S. Patent Application Ser. No. 63/016,868, filed Apr. 28, 2020 and titled “SYSTEMS AND METHODS FOR DIAGNOSING RAPID KIDNEY FUNCTION DECLINE,” each of which is incorporated herein by reference in its entirety.

This invention was made with government support under R01DK096549 awarded by National Institute of Diabetes and Digestive and Kidney Diseases and under K23DK107908 awarded by National Institute of Health. The government has certain rights in the invention.

### TECHNICAL FIELD

The present disclosure relates to the field of artificial intelligence/machine learning, and in particular to apparatus and methods for training and using machine learning models for diagnosis of kidney function decline.

### BACKGROUND

Approximately 1 out of 4 adults with diabetes has kidney disease (i.e., Diabetic Kidney Disease or DKD) and approximately 50,000 individuals will progress to kidney failure resulting in either dialysis or kidney transplants every year. The reality is that many primary care providers (PCP) and/or diabetologists are not aware, cannot adequately diagnose/prognosticate, or do not act upon declining kidney function until too late. Thus, a need exists for improved reliable risk assessment apparatus and methods in the pre-dialysis setting.

The lack of appropriate prognostic tools and the resultant delay in care with a nephrology specialist is a significant contributing factor for the high incidence of progressive decline in kidney function (e.g., rapid kidney function decline (RKFD)), and ultimately kidney failure, in this population. Standard clinical measurements of kidney function, i.e. estimated glomerular filtration rate (eGFR) and urinary albumin creatinine ratio (uACR), which are incorporated into the Kidney Disease: Improving Global Outcomes (KDIGO) guidelines for risk stratification are often not very effective for identifying those patients that will experience rapid kidney function decline especially in the early stages of DKD (G1-G2, A2-A3, or G3a-G3b). As a result, many patients are not managed effectively and their primary care provider and/or diabetologist is unaware of the progressive nature of their disease, which may result in a high proportion (25-50%) of patients starting unplanned dialysis.

Moreover, individuals with African ancestry have higher rates of end-stage kidney disease (ESKD) compared with European Americans across all baseline eGFR levels. Of relevance, genetic studies demonstrate that two distinct alleles in the Apolipoprotein L1 (APOL1) gene confer increased risk for many kidney diseases in those with African ancestry. The APOL1 high-risk (APOL1-HR) genotypes (i.e., two copies of risk allele) are associated with increased risk of ESKD, CKD incidence/progression, and eGFR decline. Although the individuals with African ancestry populations are on average at higher risk than the general population, accurate prediction of who will have progressive decline in kidney function, such as an eGFR decline >5 ml/min per 1.73 m² per year, and worse kidney outcomes is lacking. A current standard for ESKD prediction in CKD stages 3-5 is the Kidney Failure Risk Equation, where clinical variables are assigned standard weights for a recursive score calculation. The Kidney Failure Risk Equation, however, has not been validated in individuals with relatively preserved kidney function at baseline.

Accordingly, there is a need to improve risk-stratification of patients with early stage DKD, to allow for more appropriate patient management including referral to a nephrology specialist, increased monitoring, improved awareness of overall kidney health, and guidance towards more targeted, intensive therapies to slow the progression of diabetic kidney disease.

### SUMMARY

In some embodiments, a non-transitory processor-readable medium can store code to be executed by a processor of a first compute device. The code can include code to cause the processor to receive, from a second compute device remote from the first compute device, a trained machine learning model. The code can include code to cause the processor to receive biomarker data and HSD of a diabetic human subject. The biomarker data can indicate a level of at least one of the following biomarkers: sTNFR-1, sTNFR-2, KIM-1, and ratios to one another of any of the preceding. The HSD can include a metabolic factor, a health-related factor, or a demographic-related factor. The code can include code to cause the processor to execute the trained machine learning model to generate an indication of whether the diabetic human subject will experience a progressive decline in kidney function over a period of time.

### BRIEF DESCRIPTION OF THE DRAWINGS

FIG. 1 is a schematic description of a diagnosis device, according to an embodiment.

FIG. 2 is a flowchart showing a method of rapid diagnosis of progressive decline in kidney function over a period of time in a diabetic human subject having chronic-kidney-disease (CKD), according to an embodiment.

FIG. 3 is a flowchart showing a method of progressive decline in kidney function over a period of time in a diabetic human subject having CKD, according to an embodiment.

FIG. 4 is a table of characteristics of a complete training and validation cohorts, according to an embodiment.

FIG. 5 is table of a kidney-diagnostic algorithm (KDA) in a training cohort, according to an embodiment.

FIG. 6 is table showing a performance of the KDA in a complete training set, according to an embodiment.

FIG. 7 is an outline of pre-defined success criteria for an independent validation in a validation set, according to an impediment.

FIG. 8 shows output of validation testing, according to an embodiment.

FIG. 9 is a summary table showing primary endpoints of a validation testing, according to an embodiment.

FIG. 10 is a graph showing risks of the composite kidney events increasing by quantiles of KDA scores, according to an embodiment.

FIG. 11 is a table showing risks of the composite kidney events increasing by quantiles of KDA scores, according to an embodiment.

FIGS. 12A and 12B are graphs of a training cohort and a validation cohort, according to an embodiment.

FIG. 13 is a table showing additional risk cutoffs by potentially relevant proportions of a population, according to an embodiment.

FIG. 14 is a table showing net reclassification from Kidney Disease Improving Global Outcomes (KDIGO) risk stratification to risk stratification of a diagnosis device in training and validation, according to an embodiment.

FIGS. 15A and 15B are graphs showing illustrating Kaplan-Meier curves by risk stratification for an endpoint of sustained 40% decline in an estimated glomerular filtration rate (eGFR) or kidney failure in training or validation, according to an embodiment.

FIG. 16 is table showing baseline characteristics of participants of a trial program with baseline samples analyzed, according to an embodiment.

FIG. 17 is a table showing composite kidney outcomes by a diagnosis device vs KDIGO, according to an embodiment.

FIG. 18 is a table showing change in risk value generated by a diagnosis device from baseline to year 1 and association with a composite kidney outcome, according to an embodiment.

FIG. 19 is a graph showing cumulative incidence of a composite kidney outcome by continuous risk values generated by a diagnosis device, according to an embodiment.

FIG. 20 is a table showing absolute and relative risk for kidney outcomes by a diagnosis device vs KDIGO, according to an embodiment.

FIG. 21 is a table showing effects of canagliflozin on kidney outcomes by baseline risk values generated by a diagnosis device and KDIGO risk strata, according to an embodiment.

FIG. 22 is a graph showing absolute effect of canagliflozin vs. placebo on eGFR slope by risk value generated by a diagnosis device, according to an embodiment.

FIG. 23 is a graph showing absolute effect of canagliflozin vs. placebo on eGFR slope by KDIGO risk strata, according to an embodiment.

FIG. 24 is a graph showing changes in risk value generated by a diagnosis device over time in canagliflozin versus placebo-treated participants, according to an embodiment.

FIG. 25 is a flowchart showing study design and participant selection for analysis, according to an embodiment.

FIG. 26 is a graph showing relative feature importance, according to an embodiment.

FIG. 27 is a graph showing changes in risk for composite kidney outcome per risk value generated by a diagnosis device, according to an embodiment.

FIG. 28 is a table showing baseline characteristics of cohorts, according to an embodiment.

FIGS. 29A and 29B are graphs showing that stratification with predicted risk derived from a diagnosis device classified more patients correctly for a composite kidney end point than stratification with predicted risk derived from a clinical model in both type 2 diabetes (T2D) or APOL1-HR genotype population, according to an embodiment.

FIG. 30 is a table showing area under the receiver operator characteristic curve (AUCs) for a diagnosis device versus clinical model for subgroups, according to an embodiment.

FIG. 31 is a table showing thresholds of a diagnosis device for the composite kidney end point with sensitivity, specificity, positive predicted values/negative predicted values (PPV/NPV) for T2D and APOL1-HR populations in high- and low-risk strata, according to an embodiment.

FIGS. 32A and 32B are graphs showing patients with T2D or APOL1-HR classified as high-risk by a diagnosis device, experienced faster progression to end point of sustained 40% decline in eGFR or kidney failure, according to an embodiment.

FIG. 33 is a graph showing a SHapley Additive exPlanations (SHAP) plot for feature importance in T2D population, according to an embodiment.

FIG. 34 is a graph showing a SHAP plot for feature importance in APOL1-HR population, according to an embodiment.

FIGS. 35A and 35B are graphs showing observed and expected plots with 95% confidence interval in patients with T2D and APOL1-HR Genotypes, respectively, according to an embodiment.

FIG. 36 is a table showing characteristics in training and test datasets in T2D cohort, according to an embodiment.

FIG. 37 is a table showing characteristics in training and test datasets in APOL1-HR, according to an embodiment.

FIG. 38 is a table showing AUCs for a random forest model with clinical features without and with plasma biomarkers and ratios in training and test cohorts, according to an embodiment.

FIG. 39 is a table showing discrimination for the individual components of a composite kidney end point, according to an embodiment.

### DETAILED DESCRIPTION

Non-limiting examples of various aspects and variations of the embodiments are described herein and illustrated in the accompanying drawings.

Risk stratification has become more imperative since data has emerged from several randomized clinical trials (RCTs) on therapies to effectively treat patients with DKD, including SGLT2 inhibitors, GLP-1 agonists, endothelin antagonists, and possibly mineralocorticoid blockade. However, adoption of the use of these new therapies is lagging especially in low-risk patients where cost of treatment and presence of adverse events are limiting factors.

Several blood-based biomarkers have been investigated to aide with the prediction of progressive DKD including the soluble tumor necrosis factors ½ (TNFR½), and the plasma kidney injury molecule-1 (KIM-1). While these biomarkers have uniformly shown association with DKD progression, independently of eGFR and uACR, implementation of accurate prognostic models which combine clinical data with these plasma biomarkers in clinical practice is lacking.

Although the biomarkers TNFR-1, TNFR-2, and KIM-1 are often listed as biomarkers used in diagnosis devices and methods used herein, it should be understood that, in some embodiments, other biomarkers in the tumor-necrosis factor (TNF) superfamily, markers of fibrosis, urinary markers (e.g., Urinary epidermal growth factor (uEGF)), and/or the like can be used.

Standard statistical approaches are inadequate to fully leverage the electronic medical record (EMR) due to the number and complexity of features, nonaligned nature of data, and complex correlation structure. However, at least some contemporary supervised machine learning approaches have the capacity to combine biomarkers and longitudinal EMR data to produce predictive risk scores for specific outcomes. A simple risk score that improves the ability to identify patients with DKD at low, intermediate and high risk of rapid kidney function decline could lead to more appropriate allocation of resources, improved clinical workflow at the primary care provider level and more judicious use of medications.

Described herein are diagnosis devices and methods that are suitable for predicting progression of prevalent diabetic kidney disease (DKD) in a diabetic human subject. In particular, diagnosis devices and methods described herein can generate a risk value that the diabetic human subject will experience progressive decline in kidney function over a period of time, based on biomarker data and human subject data (HSD) of the diabetic human subject. Progressive decline in kidney function can be based upon an estimated glomerular filtration rate (eGFR) slope over the period of time, where the slope can include an eGFR decline of ≥5 ml/min/1.73 m2/year or ≥40% sustained decline or kidney failure (sustained (eGFR) <15 or long-term dialysis or kidney transplant.

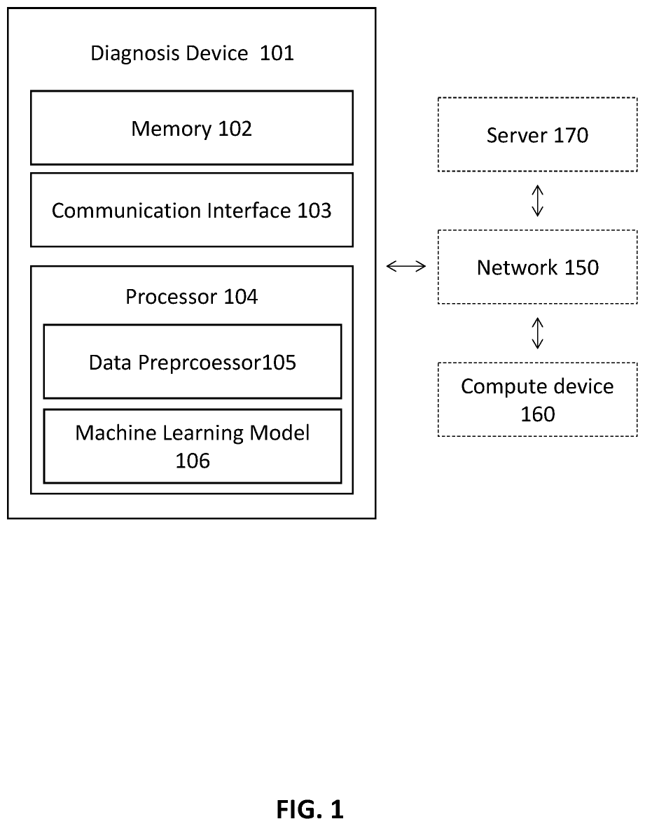

FIG. 1 is a schematic description of a diagnosis device 101, according to an embodiment. The diagnosis device 101 includes hardware and/or software to perform (or execute) a data preprocessor 105 and/or a machine learning model 106 that collectively generate a risk value that a diabetic human subject will experience progressive decline in kidney function (e.g., a rapid kidney function decline (RKFD)) over a period of time, based on biomarker data and human subject data (HSD) of that diabetic human subject. In some instances, the diagnosis device 101 can be a personal device that is in physical contact with a user. For example, the diagnosis device 101 can be, include, or be integrated to a mobile phone, a personal assistant device, and/or any device that the user can carry. In some instances, the diagnosis device 101 can be a device used by a physician. For example, the diagnosis device 101 can be, include, or be integrated to a personal assistant device of a physician, a computer of a healthcare provider, a compute device operatively coupled to a laboratory equipment, and/or the like. The diagnosis device 101 can be operatively coupled to a compute device 160 and/or a server 170 to transmit and/or receive data and/or analytical models via a network 150. The compute device 160 and the server 170 each can be/include a hardware-based computing device and/or a multimedia device, such as, for example, a server, a workstation, a computer, a desktop, a laptop, a smartphone, a tablet, a wearable compute device, and/or the like.

The diagnosis device 101 includes a memory 102, a communication interface 103, and a processor 104. In some embodiments, the diagnosis device 101 can receive data including biomarker data of a set of diabetic human subjects and/or HSD of the set of diabetic human subjects from a data source. The data source can be or include, for example, a biobank server (not shown), an external hard drive (not shown) operatively coupled to the diagnosis device 101, the compute device 160, the server 170, and/or the like. In some embodiments, the diagnosis device 101 can be configured to generate the biomarker data and the HSD, and/or the like.

The biomarker data of the set of diabetic human subjects can indicate a level of at least one of the following biomarkers: sTNFR1, sTNFR2, KIM-1, and ratios to one another of any of the preceding. The HSD of the set of diabetic human subjects can include, for example, a metabolic factor, a health-related factor, or a demographic-related factor. The metabolic factor can include, for example, a Serum Albumin level, a Serum Calcium level, a liver enzyme (AST) level, a Platelet Count, a Hemoglobin-A1C level, a Urine Albumin-Creatinine Ratio (UACR), a Glomerular Filtration Rate (ml/min), a low density lipoprotein cholesterol level, a high density lipoprotein cholesterol level, a triglyceride level, a systolic blood pressure value, or a diastolic blood pressure value, and/or the like. The health-related factor can include, for example, a body-mass-index (BMI) value, a status of past smoking, a status of current smoking, and/or the like. The demographic-related factor can include, for example, age, gender, race, ethnicity, income, education, employment history, and/or the like.

In some instances, the biomarker data of the set of diabetic human subjects can be collected, for example, from blood samples obtained at baseline, 52, 156, and 312 weeks after randomization. Biomarkers plasma TNFR-1, TNFR-2, and KIM-1 can be measured using, for example, a high performance electrochemiluminescence immunoassay on the Mesoscale Sector s600 instrument (MSD). The Biomarkers can be measured during a specific period and data associated with the biomarkers can be timetabled. In one example, the mean (minimum, maximum) CV % can be as follows: TNFR-1: 2% (0%, 10%); TNFR-2: 2% (0%, 12%); and KIM-1: 3% (0%, 18%).

In some instances, the biomarker data and/or the HSD can be from multiple geographical population (e.g., New York, San Francisco, etc.). For example, the set of diabetic human subjects can be from a first population from a first geographical location and a second population from a second geographical location. In some instances, the biomarker data and/or the HSD can be from multiple healthcare setting (e.g., a hospital, an online healthcare provider service, a pharmacy, and/or the like). For example, the set of diabetic human subjects can be from a third population from a first healthcare setting and a fourth population from a second healthcare setting.

The memory 102 of the diagnosis device 101 can be, for example, a memory buffer, a random-access memory (RAM), a read-only memory (ROM), a hard drive, a flash drive, a secure digital (SD) memory card, and/or the like. The memory 102 can store, for example, the biomarker data, the HSD, the model(s), and/or code that includes instructions to cause the processor 104 to perform one or more processes or functions (e.g., a data preprocessor 105 and/or a machine learning model 106). The communication interface 103 of the diagnosis device 101 can be a hardware component of the diagnosis device 101 to facilitate data communication between the diagnosis device 101 and external devices (e.g., the compute device 160, the server 170, peripheral devices, and/or the like). The communication interface 103 can be operatively coupled to and used by the processor 104 and/or the memory 102. The communication interface 103 can be or include, for example, a network interface card (NIC), a Wi-Fi® module, a Bluetooth® module, and/or the like. In some instances, the communication interface 103 can facilitate receiving or transmitting the biomarker data, the HSD, the machine learning model 106 (e.g., after training), and/or the like through the network 150 from/to the compute device 160 or the server 170, each communicatively coupled to the diagnosis device 101 via the network 150.

The processor 104 can be, for example, a hardware based integrated circuit (IC) or any other suitable processing device configured to run or execute a set of instructions or a set of codes. For example, the processor 104 can include a general-purpose processor, a central processing unit (CPU), a graphics processing unit (GPU), a neural network processor (NNP), and/or the like. The processor 104 can be operatively coupled to the memory 102 and/or communication interface 103 through a system bus (for example, address bus, data bus, and/or control bus; not shown). The processor 104 includes a data preprocessor 105 and a machine learning model 106. Each of the data preprocessor 105 and the machine learning model 106 can include software stored in the memory 102 and executed by the processor 104. For example, a code to cause the machine learning model 106 to generate a risk value can be stored in the memory 102 and executed by the processor 104.

The data preprocessor 105 can receive and prepare data including biomarker data and/or human subject data (HSD) of a set of diabetic human subjects (also referred to herein as ‘set of diabetic human training subjects’). The biomarker data can include a respective set of biomarker data from a biological sample collected from the respective human training subject at a respective first time. The HSD can include a respective first set of human subject data (HSD) collected from the respective human training subject at the respective first time. The HSD can further include a respective second set of HSD collected from the respective human training subject at a respective second time after the respective first time. The data preprocessor 105 can harmonize the data to improve or speed-up training of the machine learning model 106. In some instances, race or ethnicity data from the HSD can be classified into 4 major nonoverlapping categories (e.g., White, non-Hispanic Black, Hispanic, and/or the like). In some instances, the HSD of the set of diabetic human subjects can include Related Health Problems (ICD) codes or Current Procedures Terminology (CPT) codes, each ICD code from the ICD codes or each CPT code from the CPT codes can be associated with a Boolean variable (e.g., yes/no) and a timestamp. In some instances, medication data from the HSD can be mapped to RxNorm codes (e.g., https://www.nlm.nih.gov/research/umls/rxnorm or https://healthdata.gov/dataset/rxnorm, the entire disclosure of each is hereby incorporated by reference in its entirety) and laboratory values to Logical Observation Identifiers Names and Codes (LOINC) codes. In some instances, missing Urine Albumin-to-Creatinine Ratio (uACR) values can be imputed to a preset value (e.g., 10 mg/g). In some instances, only variables with representation above a preset value (e.g., >70%) throughout the combined dataset (except uACR and blood pressure due to established importance in DKD) can be included and used in the diagnosis device 101. In some instances, the data preprocessor 105 can further normalize the biomarker data and/or the HSD to a common scale (same file format, same physical units, and/or the like) for analyzing the data in a cost efficient and accurate manner.

The data preprocessor 105 can be configured to perform an outcome assessment/ascertainment. In some instances, eGFR can be determined using at least one the Chronic Kidney Disease Epidemiology Collaboration (CKD-EPI) creatinine equation, the Modification of Diet in Renal Disease (MDRD) Study equation, or the Cystatin C (CysC) equation. In some instances, a linear mixed model can be employed with an unstructured variance-covariance matrix and random intercept and random slope for each diabetic human subject from the set of diabetic human subjects, to estimate a slope from a set of slopes. For example, the linear mixed model can be:

eGFRi(t)=b₀+b₀ᵢ+(b₁+b₁ᵢ)*t+eᵢ

where t is time, b₀ and b₁ are a fixed intercept and slope, respectively, and b₀ᵢ and b₁ᵢ are a random intercept and slope, respectively. The primary composite outcome can be defined as progressive decline in kidney function, or more specifically rapid kidney function decline (RKFD), incorporating eGFR slope (decline of ≅5 ml/min/1.73 m2/year), a decline in eGFR of ≥40% from baseline that is sustained (confirmed with a second eGFR also satisfying a 40% decline at least 3 months later or “kidney failure” defined by sustained eGFR<15 confirmed at least 30 days later, or receipt of long-term maintenance dialysis or receipt of a kidney transplant). In some instances, a number of (e.g., two) nephrologists can independently adjudicate all outcomes examining each individual patient over their longitudinal course, accounting for eGFR changes (ensuring sustained decline of ≥5 ml/min or ≥40% sustained decrease), indicating ICD/CPT codes and medications to ensure that outcomes represented true decline rather than a context dependent temporary change (e.g., due to medications/hospitalizations). In some instances, events occurring after a window of assessment (e.g., less than 10 years, less than 9 years, less than 8 years, less than 7 years, less than 6 years, less than 5 years, less than 4 years, less than 3 years, less than 2 years, or less than 1 year) can be considered not valid events as they are outside the window of assessment.

The diagnosis device 101 can be configured to determine, for each respective human training subject in the plurality of diabetic human training subjects, a respective indication of whether the respective human training subject experienced progressive decline in kidney function based on at least the respective second set of HSD. The machine learning model 106 (e.g., a random forest model) can be configured to be trained based on (a) the biomarker data, the HSD, a set of features derived from the biomarker data collected at the respective first time, and/or a set of features derived from the respective first set of HSD collected at the respective first time (e.g., using a feature extractor; not shown), and (b) a respective indication of whether the respective human training subject experienced progressive decline in kidney function. The machine learning model 106 can therefore be iteratively optimized to generate the respective indication of whether the diabetic human subject will experience a progressive decline in kidney function over a period of time based on the biomarker data, the HSD, the set of features derived from the biomarker data collected at the respective first time, and/or the set of features derived from the respective first set of HSD collected at the respective first time. The indication can be a probability, a likelihood, a normalized risk score, a binary status, and/or the like.

In some instances, the machine learning model 106 can determine a set of relationships between (i) a plurality of features derived from at least the set of biomarker data and the first set of HSD and (ii) an indication of whether a diabetic human will experience progressive decline in kidney function over a period of time. In some instances, the relationship from the set of relationships can indicate a trend (e.g., a periodic trend, an increase, a decrease, and/or the like) that a human will develop progressive decline in kidney function over a period of time.

In some instances, the biomarker data and the HSD of the set of diabetic human subjects can include larger biomarker data and HSD from a number of human subjects such that the trained machine learning model is generalizable. For example, the set of diabetic human subjects can include over a hundred human subjects, over a thousand human subjects, over a million human subjects, and/or the like. That is, in some embodiments, the training data set includes biomarker data and HSD for each of at least 100 training subjects (e.g., diabetic human subjects), for each of at least 500 training subjects, for each of at least 1000 training subjects, for each of at least 5000 training subjects, for each of at least 10,0000 training subjects, for each of at least 50,000 training subjects, for each of at least 100,000 training subjects, or more.

In some embodiments, the machine learning model is trained against, for each respective training subject in the plurality of training subjects, a set of at least five features, e.g., selected from the types of biomarker data and/or the human subject data (HSD) described herein. In some embodiments, the machine learning model is trained against, for each respective training subject in the plurality of training subjects, a set of at least 6, 7, 8, 9, 10, 11, 12, 13, 14, 15, 16, 17, 18, 19, 20, 25, 30, or more features.

In some embodiments, the set of features used to train the machine learning model includes at least an abundance value for sTNFR-1, an abundance value for sTNFR-2, and an abundance value for KIM-1, a comparison of (i) an abundance value for KIM-1 and (ii) an abundance value for sTNFR-I (e.g., a ratio of the abundance values), a comparison of (i) an abundance value for sTNFR-2 and (ii) an abundance value for sTNFR-I (e.g., a ratio of the abundance values), a Urine Albumin-Creatinine Ratio (UACR), a serum calcium level, a Hemoglobin-AlC level, a systolic blood pressure, a Glomerular Filtration Rate (e.g., eGFR), an Aspartate Aminotransferase (AST) level, and a platelet count.

In some embodiments, the set of features used to train the machine learning model includes at least five features selected from an abundance value for sTNFR-I, an abundance value for sTNFR-2, and an abundance value for KIM-1, a comparison of (i) an abundance value for KIM-1 and (ii) an abundance value for sTNFR-I (e.g., a ratio of the abundance values), a comparison of (i) an abundance value for sTNFR-2 and (ii) an abundance value for sTNFR-I (e.g., a ratio of the abundance values), a Urine Albumin-Creatinine Ratio (UACR), a serum calcium level, a Hemoglobin-AlC level, a systolic blood pressure, a Glomerular Filtration Rate (e.g., eGFR), an Aspartate Aminotransferase (AST) level, and a platelet count.

In some embodiments, the set of features used to train the machine learning model includes at least 6, at least 7, at least 8, at least 9, at least 10, or at least 11 features selected from an abundance value for sTNFR-I, an abundance value for sTNFR-2, and an abundance value for KIM-1, a comparison of (i) an abundance value for KIM-1 and (ii) an abundance value for sTNFR-I (e.g., a ratio of the abundance values), a comparison of (i) an abundance value for sTNFR-2 and (ii) an abundance value for sTNFR-I (e.g., a ratio of the abundance values), a Urine Albumin-Creatinine Ratio (UACR), a serum calcium level, a Hemoglobin-A1C level, a systolic blood pressure, a Glomerular Filtration Rate (e.g., eGFR), an Aspartate Aminotransferase (AST) level, and a platelet count.

In some embodiments, the machine learning model 106 can be or include a random forest model, a least absolute shrinkage and selection operator (LASSO) model, an eXtreme Gradient Boosting (XGBoost) model, a support vector machine (SVM), an artificial neural network (ANN) model, a fully connected neural network model, a deep learning model, and/or the like.

The machine learning model 106 can include a set of model parameters such as a weight(s), a bias(s), or an activation function(s) that can be iteratively optimized to improve accuracy of predicting indications (e.g., risk value) that the set of diabetic human subjects will experience decline in kidney function over the period of time, based on the biomarker data and the HSD of the set of diabetic human subjects. Once trained, the machine learning model 106 can be executed to receive biomarker data and/or HSD about a diabetic human subject not included in the set of diabetic human subjects, and perform arithmetic calculations on the biomarker data, HSD, the weights, the biases, or the activation functions to calculate an indication (e.g., a risk value) that the diabetic human subject not included in the set of diabetic human subjects develop progressive decline in kidney function.

In some embodiments, the trained machine learning model includes at least 50 weights. In some embodiments, the trained machine learning model includes at least 100, at least 150, at least 200, at least 300, at least 400, at least 500, at least 750, at least 1000, at least 2500, at least 5000, at least 10,000, at least 15,000, at least 20,000, at least 25,000, at least 50,000, at least 100,000, or more weights.

In some embodiments, the trained machine learning model is a random forest algorithm. In some embodiments, the random forest includes at least 5 trees, at least 10 trees, at least 20 trees, at least 50 trees, at least 100 trees, at least 250 trees, at least 500 trees, or more trees.

In some instances, the biomarker data and the HSD can be split into a training dataset and a validation dataset with a preset proportions (e.g., 60% training-40% validation, 70% training-30% validation, 50% training-50% validation, and/or the like). In some instances, the machine learning model 106 can be a random forest model and configured to conduct a multi-fold cross-validation (e.g., a 10-fold cross-validation) over the set the biomarker data and the HSD. The random forest model can include a set of hyperparameters. For example, the set of hyperparameters can include a first hyperparameter associated with a number of decision trees in the forest, a second hyperparameter associated with a number of variables randomly selected for splitting at each node, and/or a third hyperparameter associated with minimum size of terminal nodes. The diagnosis device 101 can iteratively execute the random forest model based on the biomarker data and HSD of the set of diabetic human subjects to tune each hyperparameter from the set of hyperparameters. Once trained, the random forest model can be executed to receive biomarker data and/or HSD about a diabetic human subject not included in the set of diabetic human subjects, and perform arithmetic calculations on the biomarker data, HSD, and the set of hyperparameters of the random forest model, to calculate an indication (e.g., a risk value) that the diabetic human subject not included in the set of diabetic human subjects develop progressive decline in kidney function.

In some instances, the minimum size of terminal nodes can be determined (or optimized) such that the random forest model can better represent a correlation between biomarker data and HSD, and indications of whether a diabetic human will experience progressive decline in kidney function over a period of time. For example, the minimum size of terminal nodes can include ten a hundred terminal node, a thousand terminal node, and/or the like.

The compute device 160 be/include a hardware-based computing device operatively coupled to the diagnosis device 101. The compute device 160 can be configured to transmit and/or receive data and/or analytical models to/from the diagnosis device 101. In one example, in some implementations, the compute device 160 can be a device at a healthcare facility that receives from the diagnosis device 101 and/or the server 170, via an application programming interface (API), a representation of biomarker data and/or HSD about a diabetic human subject. In another example, in some implementations, the compute device 160 can be a personal device of a diabetic human subject that transmit a representation of biomarker data or HSD about the diabetic human subject, to the diagnosis device 101 and/or the server 170, via an application programming interface (API).

The server 170 can be/include a compute device medium particularly suitable for data storage purpose and/or data processing purpose and can include, for example, a network of electronic memories, a network of magnetic memories, a server(s), a blade server(s), a storage area network(s), a network attached storage(s), and/or the like. The server 170 can include a memory 172, a communication interface 173 and/or a processor 174 that are structurally and/or functionally similar to the memory 102, the communication interface 103 and/or the processor 104 as shown and described with respect to the diagnosis device 101. In one example, the server 170 can be a biobank server that stores a large amount of biomarker data and HSD about diabetic human subjects.

The network 150 can be a digital telecommunication network of servers and/or compute devices. The servers and/or computes device on the network can be connected via one or more wired or wireless communication networks (not shown) to share resources such as, for example, data storage and/or computing power. The wired or wireless communication networks between servers and/or compute devices of the network 150 can include one or more communication channels, for example, a radio frequency (RF) communication channel(s), a fiber optic commination channel(s), an electronic communication channel(s), and/or the like. The network 150 can be, for example, the Internet, an intranet, a local area network (LAN), and/or a combination of such networks.

In some implementations, the machine learning model 106 can determine a risk value of a diabetic human subject to be within a preset range and/or the diagnosis device 101 detect the risk value is within the preset range (e.g., in an intermediate risk range). The diagnosis device 101 can then monitor and/or notify a healthcare provider to monitor the human subject for an improvement in a level of the risk score or at least one biomarker from the biomarker data or at least one factor of the HSD data of the diabetic human subject.

In some implementations, after receiving a risk value, the diagnosis device 101, the compute device 160, and/or the server 170 can be configured to estimate a cardiovascular disease risk based the risk value that the diabetic human subject develops progressive decline in kidney function over the period.

In some implementations, the diagnosis device 101, the compute device 160, and/or the server 170 can be configured to determine a risk value of a diabetic human subject to be above a preset threshold (e.g., in an high risk range) and/or the diagnosis device 101 detect the risk value is within a preset range above the preset threshold. The diagnosis device 101 can be configured to then send an alarm to a compute device (e.g., the compute device 160) associated with the diabetic human subject prior to visit a healthcare provider.

In some implementations, the diagnosis device 101, the compute device 160, and/or the server 170 can be configured to classify the diabetic human subject as a low risk patient, an intermediate risk patient, or a high-risk patient. For example, diabetic human subjects with a risk value between 5 and 45 can be classified as “low risk”, between 50 and 85 can be classified as “intermediate risk”, and those with a risk value between 90 and 100 can be classified as “high risk”. In some instances, by determining proportion experiencing the composite kidney event within the categorical risk strata and used logistic regression, the relative odds of the endpoint for high vs. low risk and intermediate risk score strata can be determined.

In some implementations, the diagnosis device 101, the compute device 160, and/or the server 170 can be configured to administer a therapy (or a treatment) to reduce the risk value that the diabetic human subject will experience progressive decline in kidney function over the period of time. The diagnosis device 101, the compute device 160, and/or the server 170 can be configured further to assess a treatment effect of the therapy by calculating a trend of risk values generated over time.

In some implantations, the diagnosis device 101 or a healthcare provider using the diagnosis device can administer a first therapy (or a first treatment) if the risk value that the diabetic human subject will experience progressive decline in kidney function over the period of time is above a pre-set threshold (e.g., top 15% in T2D and in APOL1-HR). The diagnosis device 101 or a healthcare provider can administer a second therapy (or a second treatment) if the risk value that the diabetic human subject will experience progressive decline in kidney function over the period of time is below the pre-set threshold (e.g. bottom 85% in T2D and in APOL1-HR). In one example, the first therapy can involve providing the diabetic human subject with a first dose of medication when the risk value is above the pre-set threshold and the second therapy involve providing the diabetic human subject with a second dose of medication (different from the first dose of medication) when the risk value is below the pre-set threshold. In another example, a medication (e.g., canagliflozin) for patients with eGFR decline in intermediate risk stratum (1.52 ml/min/1.73 m2) and high risk stratum (2.16 ml/min/1.73 m2) can be used while a lifestyle change therapy can be used for low risk stratum (0.66 ml/min/1.73 m2).

In some embodiments, the results obtained from application of the machine learning model to patient data stratify the patient into one of at least two risk categories for progressive decline in kidney function. In some embodiments, where the subject is not already taking diabetes medication, the methods described herein further include, when the subject is assigned to a high risk category, administering a diabetes medication to the subject and, when the subject is assigned to a low risk category, administering a lifestyle change therapy to the subject (e.g., not administering the diabetes medication to the subject.

Non-limiting examples of gliflozin Sodium-Glucose Cotransport 2 inhibitors include dapagliflozin (e.g., (2S,3R,4R,5S,6R)-2-[4-chloro-3-[(4-ethoxyphenyl)methyl]phenyl]-6-(hydroxymethyl)oxane-3,4,5-triol), empagliflozin (e.g., (2S,3R,4R,5S,6R)-2-[4-chloro-3-[[4-[(3S)-oxolan-3-yl]oxyphenyl]methyl]phenyl]-6-(hydroxymethyl)oxane-3,4,5-triol), canagliflozin (e.g., (2S,3R,4R,5S,6R)-2-[3-[[5-(4-fluorophenyl)thiophen-2-yl]methyl]-4-methylphenyl]-6-(hydroxymethyl)oxane-3,4,5-triol), and/or ertugliflozin (e.g., (1S,2S,3S,4R,5S)-5-[4-chloro-3-[(4-ethoxyphenyl)methyl]phenyl]-1-(hydroxymethyl)-6,8-dioxabicyclo[3.2.1]octane-2,3,4-triol). These, and other, gliflozin Sodium-Glucose Cotransport 2 inhibitors are described, for example, in Dekkers et al., Curr. Diabetes Reports, 18:27 (2018), the content of which is hereby incorporated by reference

In some implementations, the machine learning model 106 can be configured to generate a first indication of whether the diabetic human subject will experience a progressive decline in kidney function at a first time. The first indication can be a first probability, a first likelihood, a first normalized risk score, a first binary status, and/or the like. The diagnosis device 101 can then determine a indication of whether the diabetic human subject will experience a progressive decline in kidney function at a second period of time (after the first period of time). The second indication can be a second probability, a second likelihood, a second normalized risk score, a second binary status, and/or the like. A difference between the first indication at the first period of time and the second indication at the second period of time can be informative about a change in the diabetic human subject. In some instances, the diagnosis device 101 can be configured to further train the machine learning model 106 based on at least one the first indication or the second indication. In some instances, the diagnosis device 101 can determine the second risk value over the second period of time after administering a therapy (or treatment) on the diabetic human subject. In such instances, the difference between the first indication at the first period of time and the second indication at the second period of time can be informative about the therapy administered on the diabetic human subject. The second value at the second period of time can be informative about changes in risk for progression based on the natural history of DKD, or informative about the therapy administered on the diabetic human subject. The therapy can include, for example, a therapy based on SGLT2i (e.g. canagliflozin), a therapy based on angiotensin converting enzyme (ACE) inhibitors, or a therapy based on angiotensin-receptor blockers (ARBs). In some instances, the therapy can include a change in habits such as, for example, a change in lifestyle, a change in diet, or a change in exercise.

Although the diagnosis device 101, the compute device 160, and the server 170 are shown and described as singular devices, it should be understood that, in some embodiments, one or more diagnosis devices, one or more compute device, and/or one or more server devices can be used in a diagnosis system.

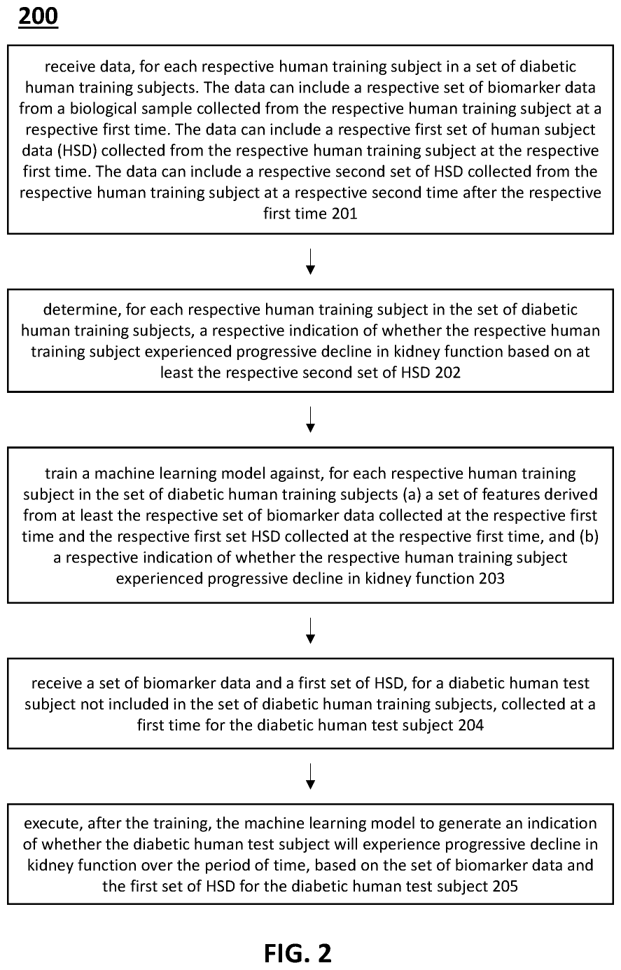

FIG. 2 is a flowchart showing a method 200 of rapid diagnosis of kidney function decline over a period of time in a diabetic human subject having chronic-kidney-disease (CKD), according to an embodiment. In some implementations, a diagnosis device (such as the diagnosis device 101 as shown and described with respect to FIG. 1) can be used to perform the method 200. At 201, the diagnosis device can receive data, for each respective human training subject in a set of diabetic human training subjects. The data can include a respective set of biomarker data from a biological sample collected from the respective human training subject at a respective first time. The data can include a respective first set of human subject data (HSD) collected from the respective human training subject at the respective first time. The data can include a respective second set of HSD collected from the respective human training subject at a respective second time after the respective first time. At 202, the diagnosis device can determine, for each respective human training subject in the set of diabetic human training subjects, a respective indication of whether the respective human training subject experienced progressive decline in kidney function based on at least the respective second set of HSD.

At 203, the diagnosis device can train a machine learning model against, for each respective human training subject in the set of diabetic human training subjects (a) a set of features derived from at least the respective set of biomarker data collected at the respective first time and the respective first set HSD collected at the respective first time, and (b) a respective indication of whether the respective human training subject experienced progressive decline in kidney function. At 204, the diagnosis device can receive a set of biomarker data and a first set of HSD, for a diabetic human test subject not included in the set of diabetic human training subjects, collected at a first time for the diabetic human test subject. At 205, the diagnosis device can execute, after the training, the machine learning model to generate an indication of whether the diabetic human test subject will experience progressive decline in kidney function over the period of time, based on the set of biomarker data and the first set of HSD for the diabetic human test subject. The machine learning model can determine a relationship between (i) a set of features derived from at least the set of biomarker data and the first set of HSD and (ii) an indication of whether a diabetic human will experience progressive decline in kidney function over a period of time. The relationship can indicate a probability or risk that a human will experience a progressive decline in kidney function over a period of time.

In some embodiments, the method 200 can further include receiving, for each respective human training subject in the set of diabetic human training subjects, a respective third set of HSD collected from the respective human training subject at a third time before the first time. In some embodiments, the method 200 can include receiving, for each respective human training subject in the set of diabetic human training subjects, a respective fourth set of HSD collected from the respective human training subject at a third time after the second time.

In some embodiments of the present disclosure, a method (which may also be referred to as a kidney-diagnostic algorithm or KDA) for determining risk of developing progressive decline in kidney function over a period of time in a diabetic human subject having chronic-kidney-disease (CKD) is provided. The method can include detecting biomarker data in a first biological sample of a diabetic human subject having CKD, the biomarker data indicating a level of at least one of the following biomarkers: sTNFR-I, sTNFR-2, KIM-1, and ratios to one another of any of the preceding. The method can further include obtaining human subject data (HSD) of the human subject. The HSD can include at least one of metabolic factors indicating detected levels in the first biological sample, or a second biological sample, of the diabetic human subject of at least one of: Serum Albumin, Serum Calcium, Serum Potassium, Serum Chloride, HDL, LDL, Triglycerides, Haemoglobin, Platelets, AST, ALT, Blood Urea Nitrogen (BUN), Red Cell Distribution (RDW), Hemoglobin-AlC, Urine Albumin-Creatinine Ratio, and Glomerular Filtration Rate (ml/min), as well as health- related factors including at least one of: body-mass-index (BMI), diastolic blood pressure, systolic blood pressure, weight, height, pulse rate, value, and demographic-related factors of the human subject indicating to demographic data of the human subject (the demographic-related data can include at least age, race/ethnicity, smoking status). The method can further include determining at least one relationship between a level, or a range of levels, of at least one of the biomarkers and a reference value, or a range of reference values, and the at least one factor of the HSD, based on training data, wherein the relationship indicates to a probability or risk that a human will develop at progressive decline in kidney function over the period of time, and determining a risk that the diabetic human subject develops progressive decline in kidney function over the period of time, based on the determined relationship.

In some instances, obtaining human subject data (HSD) of the human subject can involve measuring the HSD. For example, the diagnosis device can be operatively coupled to a measurement device that measure a set of factors of the HSD from the human subject. In some instances, obtaining the HSD can involve fetching the HSD from a compute device operatively coupled to the diagnosis device. For example, the diagnosis device can be configured to fetch the HSD from a server that is operatively coupled to the diagnosis device.

Such embodiments, may include one and/or another of (and in some embodiments, a plurality of, and in some further embodiments, all of) the following additional functionality, feature(s), structure, step(s), or clarification(s), leading to yet further embodiments of the present disclosure:

the period of time can be less than 10 years, less than 9 years, less than 8 years, less than 7 years, less than 6 years, less than 5 years, less than 4 years, less than 3 years, less than 2 years, or less than 1 year;
determining the relationship includes analyzing the training data;
the training data can also include validation data;
validating the relationship using validation data;
determining the relationship based on training data is accomplished via a machine learning;
machine learning is associated with a training model, such that:
   the model, in some embodiments, can include a random forest model,
   the random forest model can include multi-fold cross-validation using the at least one biomarker and at least one HSD factor, as data inputs; and/or
   the training model can be configured to predict a composite kidney endpoint of progressive decline in kidney function;
   the progressive decline in kidney function is based upon an estimated glomerular filtration rate (eGFR) slope over the period of time, where the slope can include an eGFR decline of ≥5 ml/min/1.73 m²/year or ≥40% sustained decline or kidney failure (sustained (eGFR) <15 ml/min/1.73 m² or long-term dialysis or kidney transplant).
administering a therapy to the human subject if the determined risk is greater than a predetermined threshold;
the risk indicates a score (which can also be referred to as a risk score); and
monitoring the human subject for a reduction or increase in the level of the risk score, at least one biomarker, and/or a change in the at least one factor of the HSD data.

In some embodiments, a system is provided, which can be configured to perform a plurality of the steps of any of the embodiments disclosed herein (see, e.g., above). In such embodiments, the system may include one or more processors having instructions operating thereon for causing the system to perform the plurality of steps.

Embodiments of the present disclosure are directed to a kidney diagnostic method, which may also be referred to, in some embodiments, as an algorithm (KDA), for determining a risk for a human subject, and in particular, a human subject having/diagnosed with diabetes (type 2) and associated diabetic kidney disease (DKD), in developing progressive decline in kidney function within a period of time (e.g., in some embodiments, within 5 years).

The prediction of progressive decline in kidney function in patients with type 2 diabetes is challenging, particularly in patients with preserved kidney function. The ability to improve risk-stratification of patients into low, intermediate and high-risk groups with the KDA, according to some embodiments, allows for more appropriate patient management including:
referral to a nephrology specialist,
increased monitoring,
improved awareness of overall kidney health, and
guidance towards more targeted, intensive therapies to slow the progression of DKD.

The KDA, according to some embodiments, includes inputs from at least one, and in some embodiments, two, and in some embodiments, three blood-based biomarkers (and ratios with respect to each of said three biomarkers with one another) that have been examined in several clinical settings, including patients with DKD. Soluble tumor necrosis factor receptor 1 and 2 and plasma kidney injury molecule-1 have demonstrated reliable independent prognostic signals for kidney function decline and ESRD. The individual biomarkers have generally added, in some embodiments, significant improvement to classical clinical metrics for prediction of progressive decline in kidney function, and the combination of all three (in such embodiments), is synergistic.

Assessment of risk in patients with DKD is currently based on categories of Urine Albumin-to-Creatinine Ratio (uACR) and estimated glomerular filtration rate (eGFR) in accordance with the 2012 Kidney Disease Improving Global Outcomes (KDIGO) Clinical Practice Guidelines for the Evaluation and Management of Chronic Kidney Disease. According to these guidelines, primarily only patients with eGFR<30 ml/min/1.73 m2 or UACR≥300 mg/g should be referred for escalation of care to a nephrologist and intensive monitoring (>3 times per year). However, this population comprises only ˜30% or 2.7 million of the total DKD patient population in the United States. The KDIGO guidelines also recommend that DKD patients with eGFR 30-59 ml/min/1.73 m2 or UACR 30-300 mg/g (70% of the overall DKD population or ˜6.6 million patients in the United States), be monitored at the PCP/diabetologist level without escalation of care to a nephrologist even though many of these patients experience rapid kidney function decline.

In some embodiments, the KDA identified 2.5-fold more patients as high risk that went on to experience DKD progression in 5 years than KDIGO very high risk-strata. Notably, this improvement was observed in the same proportion of the population (approximately 15%) categorized in the highest risk strata. This would translate into ˜1 million more events identified accurately in the US over KDIGO criteria.

The integrated approach, according to some embodiments, has several, near-term clinical implications, especially when linked to clinical decision support (CDS) and embedded care pathways within the EMR. For example, patients with a high KDA/risk score, who will have a predicted probability of >50% (and up to 62% in a validation cohort) of experiencing progressive decline in kidney function (e.g., RKFD) would be recommended to see a nephrology specialist, which has been shown to be associated with improvement in outcomes. In addition, referral to a dietician and delivery of educational materials regarding the importance and consequences of chronic kidney disease, particularly in the presence of type 2 diabetes, can be provided to patients with high KDA scores to help increase awareness and facilitate motivation for changes in lifestyles and behavior. Finally, optimization of medical therapy including renin-angiotensin aldosterone system inhibitors, statins for cardiovascular risk management, and intensification of antihypertensive medication to meet guideline recommended blood pressure targets can be pursued. The application of sodium glucose transporter (SGLT)-2 inhibitors can also be most advantageous in a high KDA/RKFD risk score group with type 2 diabetes given data on robust renoprotection with these agents. On the other hand, patients with a low predicted probability could be clinically managed by their primary care provider and have standard of care treatment with scheduled monitoring of their kidney function and KDA scores. Finally, patients with an intermediate risk score can be recommended for more frequent monitoring of standard of care and retesting longitudinally. Such patients may demonstrate score shifts based on behavior, clinical parameters and treatments change over time with appropriate clinical actions as necessary. This overall approach would not only have benefits for individual patient outcomes but also at a health system and population level, where there is inconsistency in how these patients are treated.

### EXAMPLE 1

The following is an example of use of the KDA according to some embodiments of the present disclosure.

Limitations. First, there was limited geographic distribution of the two study cohorts, both being from the Northeast portion of the United States. Despite that, the study population was diverse in terms of race and sex, and the nature of unselected enrollment from outpatient clinics at the two centers favors generalizability. Second, since these data were extracted from EMRs and reflected “real world” practice patterns, 38% of the population was missing uACR values. Accordingly, an imputation method was used for uACR that has been adopted by other large investigative groups (imputation methods were also used for missing laboratory variables).

Laboratory candidacy was limited as a feature in the models for this example, unless it was present in at least 70% of the population. Despite the need for imputation, the ability to risk stratify the patient populations succeeded. Additionally, a time-horizon of 5 years was only considered to develop the composite kidney endpoint. Kidney events that occurred after the 5-year mark were not construed as events in the model.

Accordingly, a machine learning model combining plasma biomarkers and EMR data significantly improved prediction of progressive kidney function decline and kidney failure over standard clinical models in patients with early stages of DKD from two large academic medical centers.

Candidate Data

Individuals were identified with prevalent diabetic kidney disease (DKD), from two electronic medical record (EMR)-linked retrospective plasma biobanks: Mount Sinai BioMe Biobank, NYC, N.Y. (BioMe) and the University of Pennsylvania Medicine Biobank, Philadelphia, Pa. (PMBB).

Inclusion Criteria

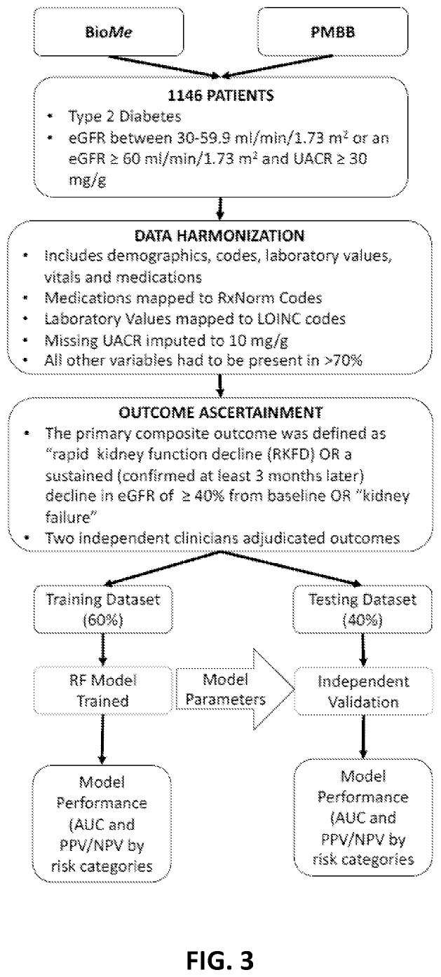

Patients were selected from BioMe and PMBB that were aged between 21-90 at the time of enrollment into the respective biobanks (“baseline”), had a diagnosis of type 2 diabetes, a baseline estimated glomerular filtration rate (eGFR) between 30 and 59.9 ml/min/1.73 m²/year or a baseline eGFR>60 with a urinary albumin to creatinine ratio (uACR)≥30 mg/g. The eGFR and uACR criteria effectively define patients with DKD stages G3a, G3b or G1/G2, A2, A3. Also, to be eligible for this study, the patient enrolled into the biobank must have had a stored plasma specimen, a minimum follow-up time after the time of enrolment of at least 21 months, and at least 3 eGFR values after baseline (FIG. 3). Patients with kidney transplant or chronic maintenance dialysis prior to date of enrollment into the biobank were excluded from the study.

Sex and race were obtained from an enrollment questionnaire administered to the biobank participants. Clinical data were extracted for all continuous variables (eGFR, hemoglobin Alc, urine albumin to creatinine ratios) at baseline from the EMR with concurrent time stamps. The baseline time point for clinical variables was one year prior to enrollment into the biobank. For eGFR, the baseline period was defined as 1 year before or up to 3 months after the biobank enrollment date, and baseline uACR values were derived from closest values +/−1 year from the date of enrollment into the biobank. Subjects without baseline values of eGFR and uACR were excluded from the study. Body mass indices (BMI) were calculated as the ratio between weight and the square of height in kg/m2. Hypertension and type 2 diabetes status at baseline were determined using the EMR and Genomics (eMERGE) Network phenotyping algorithms.16 Cardiovascular disease and heart failure were determined by a validated algorithm from the EMR and Genomics (eMERGE) network and ICD-9/10 codes respectively. Follow up time was calculated from the time of enrollment date to (a) the date at which a sustained 40% decline in eGFR was reached, (b) the date of “kidney failure” (sustained eGFR<15 ml/min confirmed 30 days later, start of long-term maintenance dialysis, or receipt of kidney transplant); or (c) censoring due to loss to follow-up prior to 5 year time period.

Biospecimen Collection

Plasma specimens were collected on the day of enrollment into BioMe or into the PMBB Biobank. All plasma samples underwent standard processing as per protocols for BioMe/PMBB and were continuously stored at −80° C. until shipment for analysis.

Biomarker Assays

Three plasma biomarkers were measured in multiplex using the Mesoscale platform (Meso Scale Diagnostics, Gaithersburg, Md., USA), which employs proprietary electro-chemiluminescence detection methods combined with patterned arrays to allow for multiplexing of assays. Each sample was run in duplicate, along with quality control samples with known low, moderate and high concentration of each biomarker on each plate. Assay precision was assessed using a panel of 7 to 8 reference samples that spanned the measurement range. The intra-assay coefficient of variation (CV) results for KIM-1, TNFR-1, and TNFR-2 were mean CV 3.9% (min 3.1%, max 4.9%), mean CV 5.4% (min 3.9%, max 8.6%), and mean CV 3.7% (min 3.1%, max 4.2%), respectively. The inter-assay coefficient of variation (CV) results for the reference samples for KIM-1, TNFR-1, and TNFR-2 were mean CV 9.9% (min 8.3%, max 12.7%), mean CV 10.1% (min 6.4%, max 18.1%), and mean CV 7.8% (min 7.2%, max 9.7%). Assays satisfied dilution linearity and were run at 1:4 dilution. Levey-Jennings plots were employed and followed the Westguard rules for re-run of samples as required. Further details of the analytical validation of the biomarker assays are provided in Analytical Validation report PL-00001. The laboratory personnel performing the biomarker assays were blinded to clinical information about the participants.

Data Harmonization

Data was harmonized across the two datasets from BioMe and PMBB. Race was collapsed into 4 major categories (White, non-Hispanic Black, Hispanic, and other). International classification of disease (ICD) and Current Procedural Terminology (CPT) codes were included as yes/no variables with a timestamp. Medications were mapped to RxNorm codes (https://www.nlm.nih.gov/research/umls/rxnorm/index.html) and laboratory values to Logical Observation Identifiers Names and Codes (LOINC) codes. In some instances, only variables with >70% representation throughout the combined dataset (except uACR and blood pressure due to established importance in DKD) were included and used for developing the KidneylntelX algorithm.

Assessment and Definition of the Kidney Endpoint

eGFR was determined using the CKD-EPI creatinine equation. Linear mixed models were employed with an unstructured variance-covariance matrix and random intercept and random slope for each individual to estimate slope. Models with fer(t)=b0+b0i+(b1+b1i)*t+ei, where t is time, b0 and b1 were the fixed intercept and slope, and b0i and b1i were the random intercept and slope, respectively. The primary composite outcome was defined as “rapid kidney function decline (RKFD)” incorporating eGFR slope (decline of ≥5 ml/min/1.73 m2/year), a decline in eGFR of ≥40% from baseline that is sustained (confirmed with a second eGFR also satisfying a 40% decline at least 3 months later or “kidney failure” defined by sustained eGFR <15 confirmed at least 30 days later, or receipt of long-term maintenance dialysis or receipt of a kidney transplant). Additionally, two nephrologists independently adjudicated all outcomes examining each individual patient over their longitudinal course, accounting for eGFR changes (ensuring sustained decline of ≥5 ml/min or ≥40% sustained decrease), indicating ICD/CPT codes and medications to ensure that outcomes represented true decline rather than a context dependent temporary change (e.g., due to medications/hospitalizations). All events occurring after 5 years were not considered valid events as there were outside the window of assessment.

Statistical Analyses

An independent statistician then randomized the complete cohort into a training (60%) and validation dataset (40%). The training and validation datasets were balanced with regards to key demographics and clinical outcome. The validation dataset was sequestered from the training dataset post randomization.

The training set was then subsequently randomized into 70% training and 30% test groups for further analyses (FIG. 3). A 10-fold cross-validation was conducted on all models in the training cohort utilizing random forest. All of the clinical and biomarker variables were evaluated by the algorithm to optimize feature selection. Further iterations of the model were conducted by tuning the individual hyperparameters. For example, hyperparameter 1 is the number of decision trees in the forest, hyperparameter 2, the number of variables randomly selected for splitting at each node and hyperparameter 3, minimum size of terminal nodes.

### Results: Characteristics of the Complete Training and Validation Cohorts

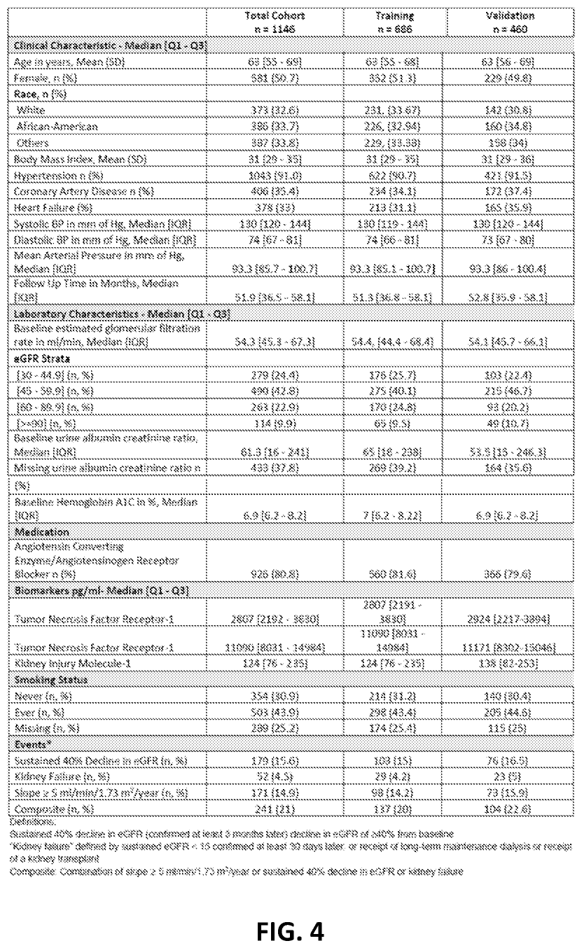

In the total cohort (n=1146), at baseline, the median age was 63 years, 51% were female, the median eGFR was 54 ml/min/1.73 m2 and the median uACR was 61 mg/g, as shown in FIG. 4. A data point for uACR was available (considered not missing within +/−1 year prior to the baseline biomarker collection) in 62% of the cohort. The most common comorbidities were hypertension (91%), coronary heart disease (35%), and heart failure (33%). The majority (81%) were on ACE inhibitors or angiotensin receptor blockers (ARBs) at baseline. Patients were randomly assigned to training and validation sets by an independent statistician with all patient characteristics including events balanced between the training and validation cohorts.

The KDA in Training Cohort

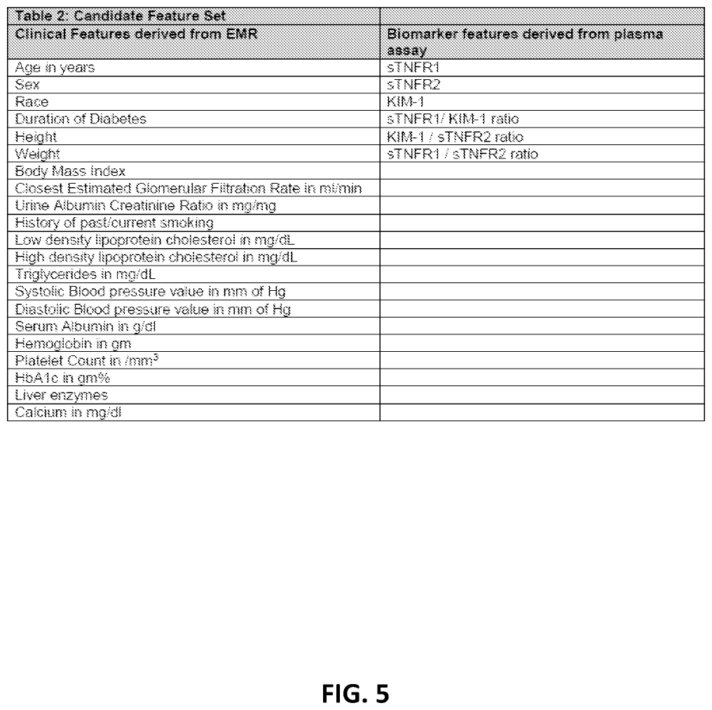

Using the complete training set (n=686) and a random forest algorithm candidate features were identified for inclusion in the KDA including ratios of the three plasma biomarkers as well as the raw concentrations and candidate clinical variables (truncated at the date of biomarker measurement). As shown in FIG. 5, missing uACR values were imputed to 10 mg/g, while multiple imputation methods were used for missing BP (using age, sex, race and antihypertensive medications). A median imputation was used for missing laboratory values. Analyses were originally performed on the training 70:30 split and then subsequently finalized on the full training cohort.

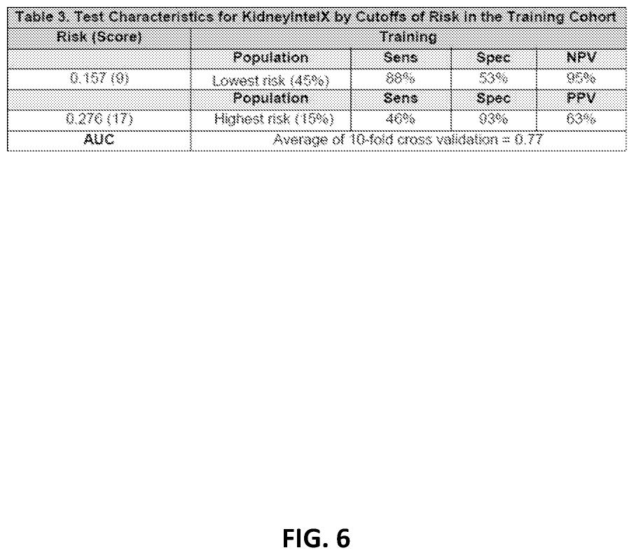

Each patient in the training set was assigned a risk score on a scale of 0-1 for the progressive decline in kidney function endpoint. The final algorithm was selected based on AUC performance and PPV/NPV derived from cut-offs representing the 15% and 45% highest and lowest risk of progressive decline in kidney function, respectively. Patients were then stratified into 20 risk score percentiles for ease of interpretation. The performance of the algorithm in the complete training set (n=686) is represented in FIG. 6. The AUC for the KDA risk model was 0.77 (95% CI 0.72-0.82). By comparison, a clinical model of eGFR and uACR yielded an AUC of 0.67 (95% CI 0.64-0.70) and the AUC for KDIGO risk strata was 0.65 (95% CI 0.63-0.70).

The KDA in Validation Cohort

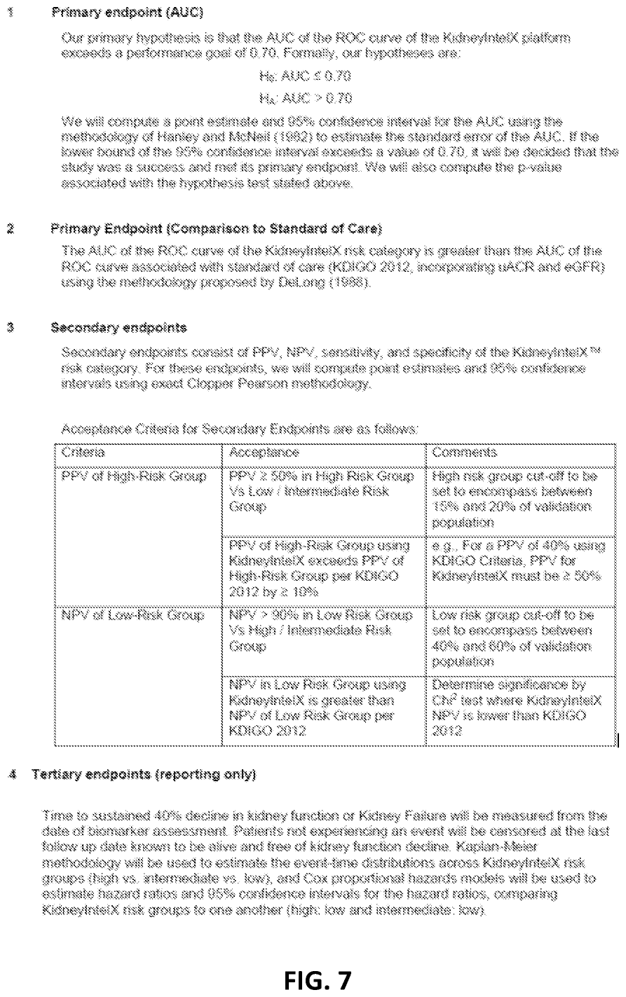

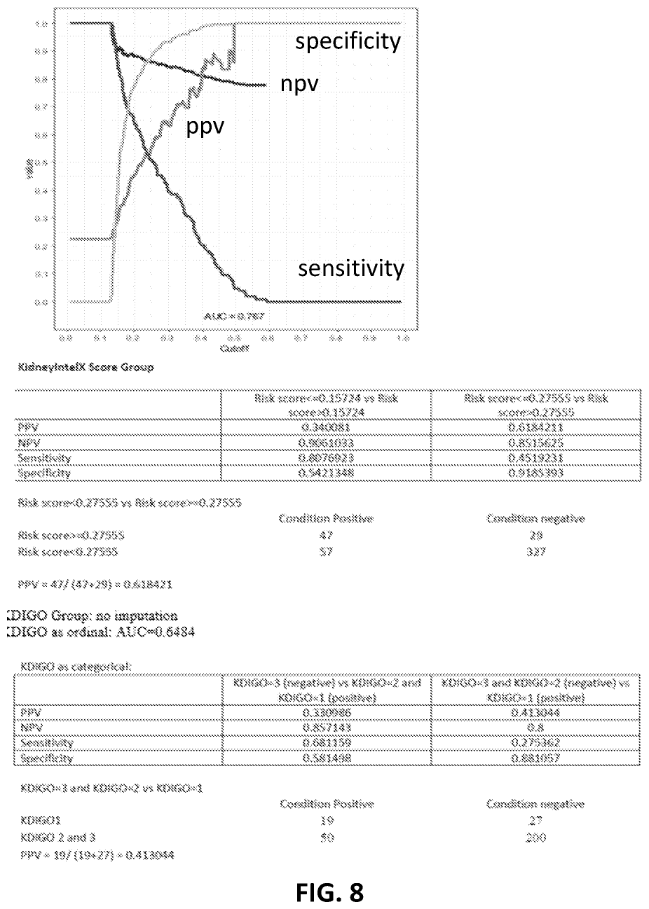

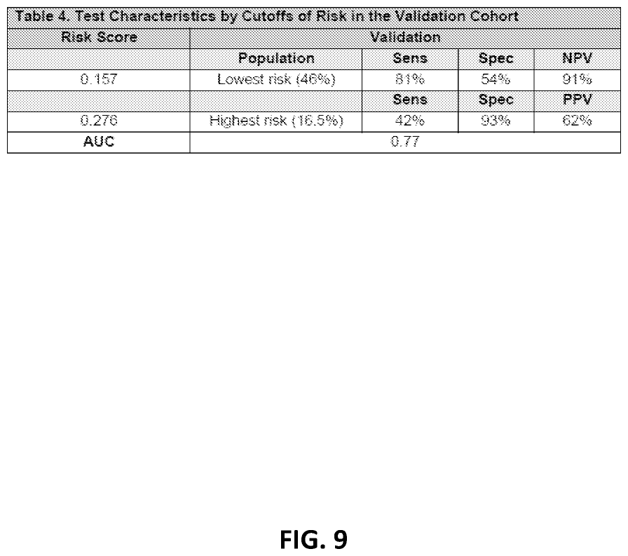

The final algorithm was brought forward in the validation cohort (n=460) and analyzed by an independent statistician (Dr. Mike Kattan, Cleveland Clinic) not previously exposed to the training data set and algorithm development. The cutoffs based on the scaled scores in the training set were applied to stratify the patients in the validation set. Success criteria for the independent validation in the validation set were pre-defined as outlined in FIG. 7, and output as shown in FIG. 8. The primary endpoints of the validation testing are summarized as follows in FIG. 9.

The AUC for the KDA as 0.77 (95% CI 0.76-0.79) in validation. By comparison, a clinical model of eGFR and uACR yielded an AUC 0.63 (95% CI 0.62-0.65) and the AUC for KDIGO risk strata was 0.63 (95% CI 0.63-0.66) in the validation set (Delong p value for the KDA vs. clinical model and the KDA vs. KIDGO, <0.001).

The KDA Risk Score and Event Probability

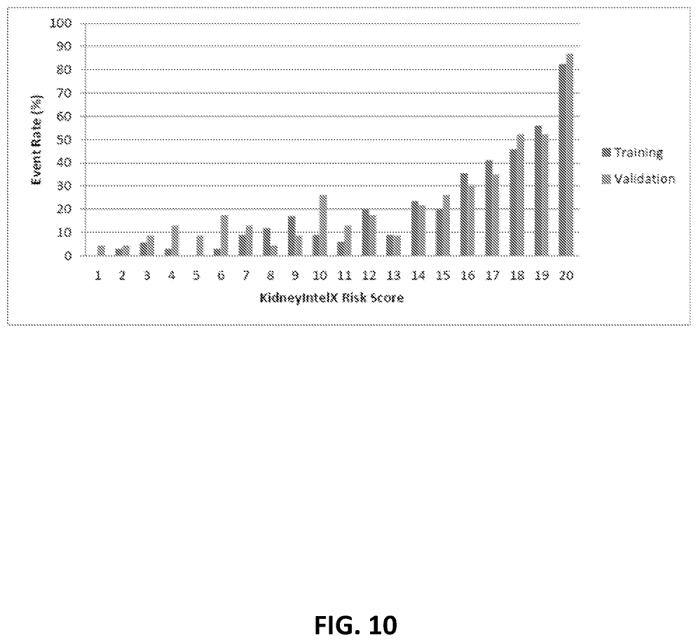

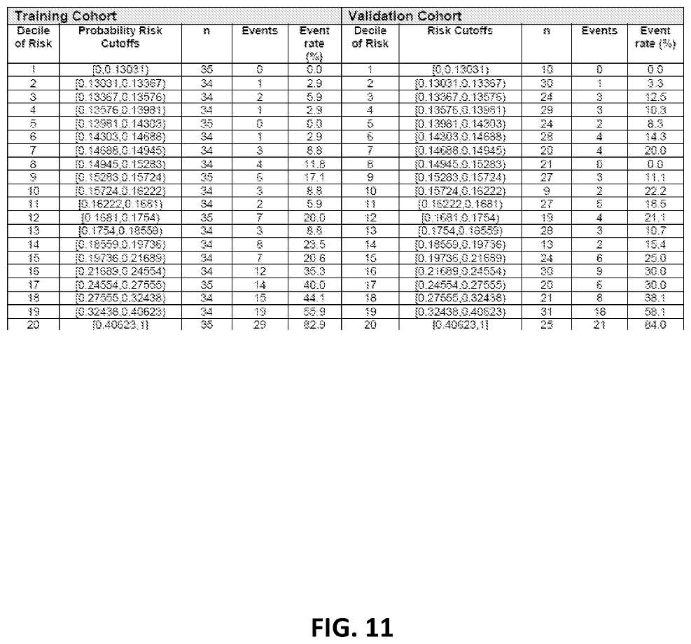

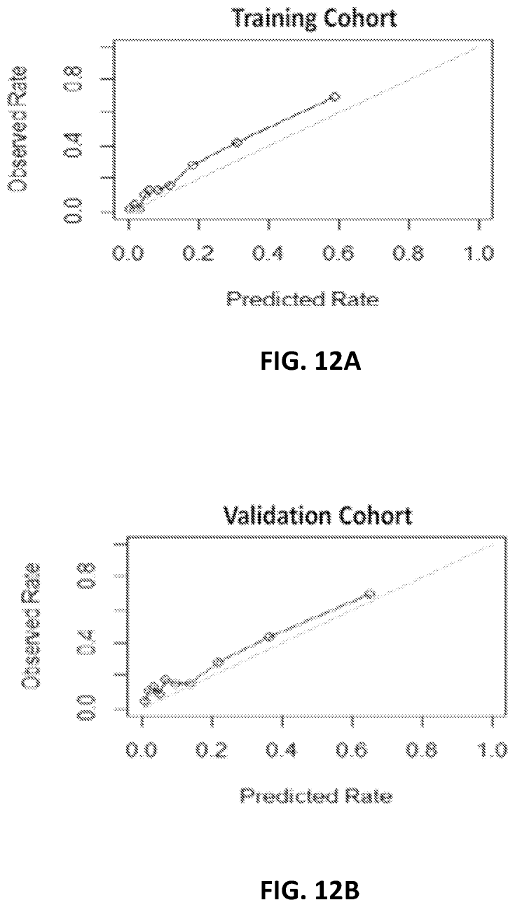

The risks of the composite kidney event increased by quantile of the KDA score are shown in FIG. 10 and FIG. 11. In addition, calibration was assessed by examination of the slope of observed vs. expected outcomes plots of the KDA score vs. the observed outcomes. The slope of the observed vs. the predicted risk for KDA was 0.8 in the training set and 1.0 in the validation set, indicating there was no statistically significant difference between the predicted and observed outcomes (FIGS. 12A-B).

KDA Clinical Validation (Additional Outcomes)

In order to further evaluate the clinical validity of the KDA, the KDA performance was compared to risk stratification outlined in KDIGO2012 utilizing eGFR and UACR. For this analysis, subjects from the validation set with available uACR (n=296), were stratified by the reference method as follows: 53%, 31% and 16% of the population into moderately increased risk, high risk, and very high risk with respective probabilities of 0.15, 0.29 and 0.41 for the composite kidney endpoint over 5 years (PPV of 41% for “very high risk” group and NPV of 85% for the “moderately high risk” group).

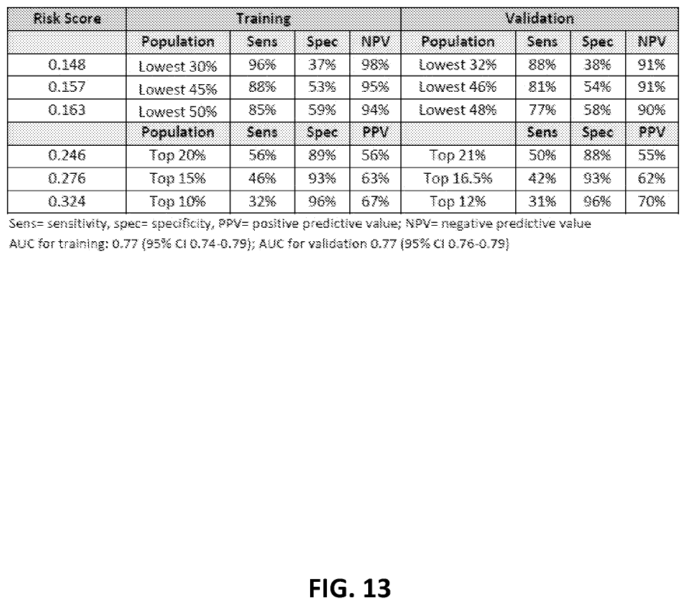

When the risk cut-offs for the KDA test were applied to the validation set with imputed uACR (n=460), KDA stratified 47%, 37% and 16.5% of patients in the cohort to low-, intermediate- and high-risk strata with respective probabilities of patients experiencing the composite kidney endpoint of 0.09, 0.22, and 0.62. This translated into a PPV of 62% in the high- risk group (compared to a PPV 41% for KDIGO “very high risk”, p value vs. KDIGO<0.001; FIG. 13). The NPV in the low-risk group was 91% for KidneylntelX algorithm (e.g., performed using the diagnosis device 101) compared to 85% for KDIGO “moderately increased risk” group (p=0.33). Additional risk cutoffs by potentially relevant proportions of the population are shown in FIG. 13.

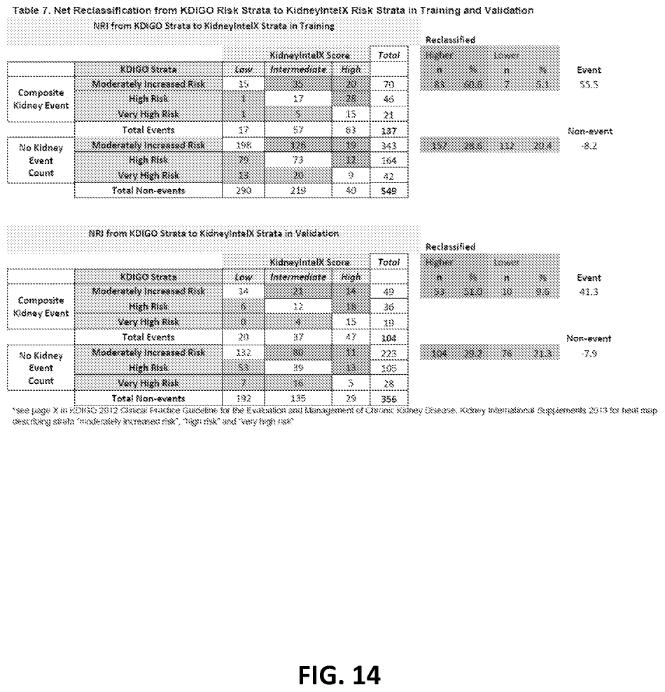

Improvement in risk classification compared to KDIGO risk strata was calculated using index of predictive accuracy (IPA)23 and net reclassification index for events and non-events. When correct classification of the outcome was compared between the KDIGO risk strata vs. KDA, the KDA test correctly classified more cases into the appropriate risk strata (NRlevent=55% in the training set and 41% in the validation set, p value<0.05; See FIG. 14). NRlnon-event was −8.2% in the training set and −7.9% in the validation set (p value NS). The integrated predictive accuracy (IPA) in the validation set was 15.2, which indicates that KDA was 15.2% more accurate than KDIGO for the composite kidney outcome.

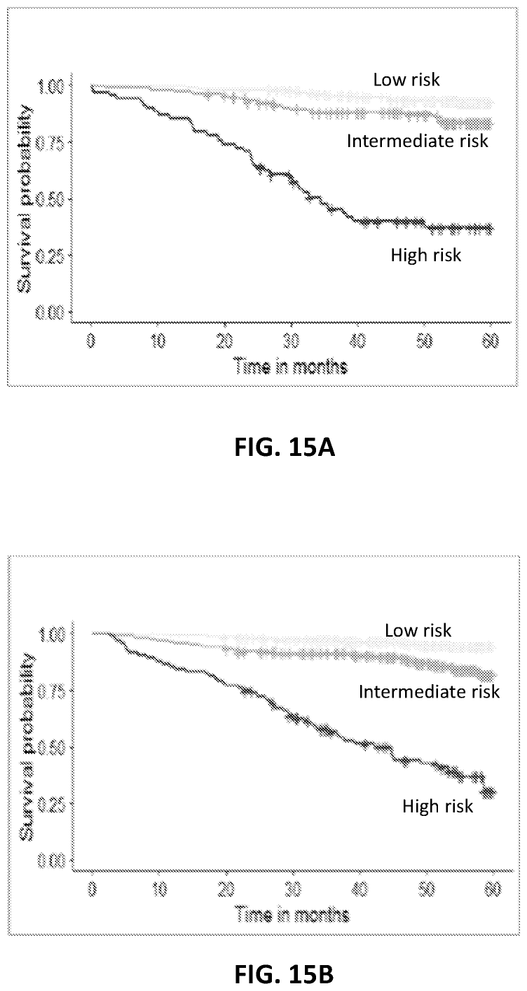

Time to Event Analyses for 40% Sustained Decline in eGFR or Kidney Failure. Patients with high-risk KDA scores (top 15% in the training set and top 16.5% in the validation set) had much higher risk of progression to the composite progressive decline in kidney function endpoint of >/=40% sustained decline in eGFR or kidney failure than patients in the low (hazard ratio (HR) 18.3; 95% CI: 10.1-33.1 in training and 14.7, 95% CI 7.8-27.6 in validation; FIG. 6). Compared to the intermediate risk strata, those in the high-risk KDX strata had approximately 6-fold greater hazards of experiencing the composite kidney event (training hazard ratio 5.7, 95% CI 3.7-8.7, validation hazard ratio 6.0 95% CI 3.5-10.0. See FIGS. 15A-B, illustrating Kaplan-Meier Curves by Risk Strata (low cutoff 45% of population, high cutoff 16% of the population) for the Endpoint of Sustained 40% Decline in eGFR or Kidney Failure in Training (FIG. 15A) and Validation (FIG. 15B).

Accordingly, in the above example, utilizing patients with type 2 diabetes from two biobanks with banked plasma samples, and linked to data from the EMR, a machine learning risk score was validated which combined clinical data and three plasma biomarkers to predict rapid kidney function decline and kidney failure over a 5-year period. The example demonstrated that the KDA model outperformed standard clinical variables for prediction of the composite kidney event, including KDIGO risk strata.

The model was also well-calibrated. There were marked improvements in discrimination over clinical models, as measured by AUC, improvements in positive predictive value by over 20% vs. KDIGO risk strata, and a modest improvement in negative predictive value. The KDA accurately identified over 40% more patients experiencing events than the KDIGO strata.

Moreover, the KDA provided good risk-stratification for the accepted FDA endpoints of sustained ≥40% decline in eGFR or kidney failure, and was able to provide approximately a 15-fold gradient between the high- risk and low-risk strata for this clinically important outcome.

CANVAS Participants and Study Design

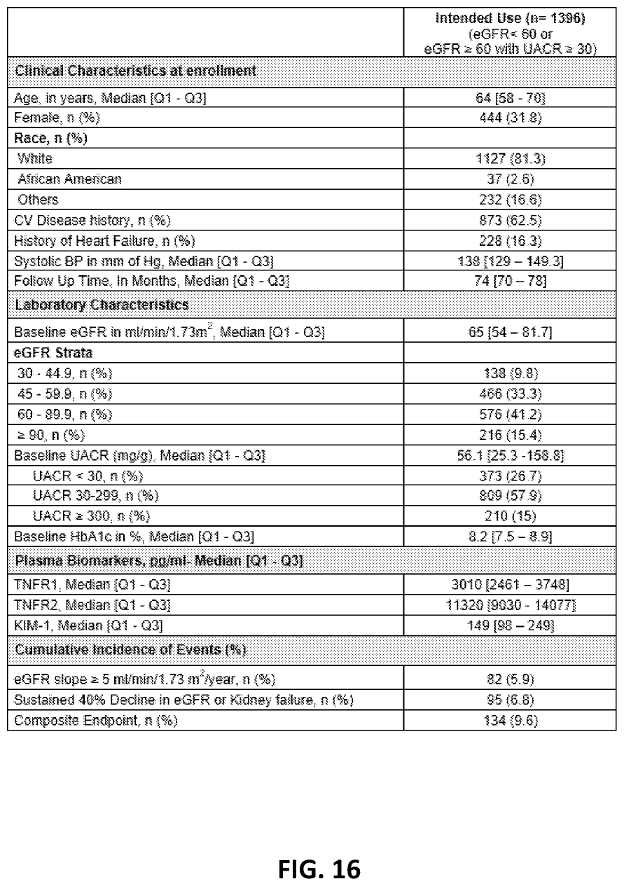

A trial program can include two multicenter, double-blinded, placebo-controlled, randomized trials (also referred to as CANVAS or CANVAS-R) to assess the effect of canagliflozin on primarily cardiovascular and secondly kidney and safety outcomes in patients with type 2 diabetes who had a history of CVD or multiple cardiovascular risk markers. Blood and urine samples for exploratory biomarker research can be stored during the CANVAS trial. In one example, the CANVAS trial enrolled 4330 participants (as shown in FIG. 16) from 24 countries. In some instances, participants can be randomly assigned using a central web-based response system in a 1:1:1 ratio to treatment with 100 mg canagliflozin, 300 mg canagliflozin, or matching placebo. In some instances, participants can be assigned to treatment with canagliflozin or placebo followed for a median of 6.1 years. All participants, care providers, trial staff, and outcome assessors can be blinded to treatment allocation for the duration of the study. KidneylntelX algorithm can be indicated for individuals with type 2 diabetes and prevalent DKD, defined by an eGFR≥30 to 59.9 ml/min/1.73 m2 (G3a and G3b) or those with eGFR≥60ml/min/1.73 m2 with a UACR≥30 mg/g to assess the risk for progressive decline in kidney function within a time period (e.g., 5 years). Thus, for analyses, the subgroup of the CANVAS population can be restricted to meet criteria for prevalent DKD at a time of enrollment.

Outcomes

A composite kidney outcome for such post hoc analysis can be defined as: A) progressive decline in kidney function (rapid kidney function decline (e.g., RKFD; with eGFR decline of ≥5 ml/min/year)); B) a sustained 40% decline of eGFR; or C) kidney failure defined as an eGFR <15 mL/min/1.73 m² or need for dialysis or kidney transplantation. In CANVAS, eGFR measurements were available at baseline, 6 weeks and annually. Since hemodynamic effects of SGLT2i on acute changes in eGFR, the anchor/baseline to define progressive decline in kidney function and 40% decline in eGFR for participants in the canagliflozin arm can be the eGFR value at 6 weeks. In some instances, when the eGFR value meet ≥40% decline from baseline only on the last eGFR obtained in the CANVAS protocol, then the event can be adjudicated by a blinded adjudication committee to determine qualification as a valid event. In some instances, eGFR slopes can be determined by linear mixed models.

Statistical Analysis

Continuous baseline variables with normal distributions can be reported as means with standard deviations (SDs). Variables with skewed distributions can be reported as median values with interquartile range (IQRs) and can be natural logarithm transformed before analysis. Furthermore, categorical variables can be reported as percentages. Risk values can be calculated by the KidneylntelX (using the diagnosis device 101) on the participants in the placebo and canagliflozin arms of CANVAS with prevalent DKD, with the inputs as per the currently commercially available test which include the following: baseline TNFR-1, TNFR-2 and KIM-1 levels, TNFR-2/TNFR-1 ratio, KIM-1/TNFR-1 ratio, eGFR, UACR, HbA1C, systolic BP, AST, platelets, and serum calcium. The risk score cutoffs that were previously validated can applied to the CANVAS participants with DKD and available baseline plasma. Each individual can be allocated a predicted probability of the composite outcome using the baseline inputs as per the risk value generated by the KidneylntelX algorithm and a continuous KidneylntelX score between 5 and 100 in increments of 5. For example, consistent with the pre-defined commercial cut offs, participants with a risk value between 5 and 45 can be classified as “low risk”, between 50 and 85 can be classified as “intermediate risk”, and those with a risk value between 90 and 100 can be classified as “high risk”. Therefore, by determining proportion experiencing the composite kidney event within the categorical risk strata and used logistic regression, the relative odds of the endpoint for high vs. low risk and intermediate risk score strata can be determined.

Proportion of participants that experienced composite kidney outcome by KidneylntelX algorithm risk strata can be compared to a proportion as classified by participants' Kidney Disease Improving Global Outcomes (KDIGO) KDIGO risk strata based on their baseline eGFR and UACR value. The odds ratios for high vs. low KidneyIntelX groups can be compared to KDIGO “very high risk” vs. “moderate risk” using the p value test for heterogeneity in a random effects weighted model. A discrimination for the composite kidney outcome for KidneyIntelX is assessed via the area under the receiver operating curve (AUC).

To explore whether baseline KidneyIntelX scores modified the treatment effect of canagliflozin versus placebo on the composite kidney outcome and chronic eGFR compared to the treatment effect as per the baseline KDIGO risk strata, tests for heterogeneity can be performed by adding interaction terms between KidneylntelX or KDIGO and randomized treatment assignment to the relevant logistic regression models for the categorical outcome and mixed linear models for the eGFR slopes. The treatment effect of canagliflozin versus placebo on KidneyIntelX risk scores over time can be assessed by calculating the difference of change in risk values generated by the KidneyIntelX algorithm (e.g., using the diagnosis device 101) between treatment arms using mixed linear models. The model included treatment allocation and time as factor and an interaction term between treatment allocation and time. The model was also adjusted for the baseline KidneyIntelX and interaction term between time and KidneyIntelX. The variance-covariance matrix was assumed to be unstructured (i.e., purely data dependent).

Associations between the 1-year change in KidneyIntelX risk scores from baseline and the composite kidney outcome was assessed using logistic regression using a landmark approach. All kidney endpoints that occurred in the first year were excluded from the analysis. The models were adjusted for baseline KidneyIntelX score and treatment arm. A test for interaction between change in KidneyIntelX and treatment arm was also performed. Finally, the proportion of participants that experienced future events in those that remained in the same risk strata at baseline to 1 year vs. those that changed risk strata over time, overall, and stratified by treatment arm, were examined. All analyses can be performed in a programming language (e.g., R).

Study Population

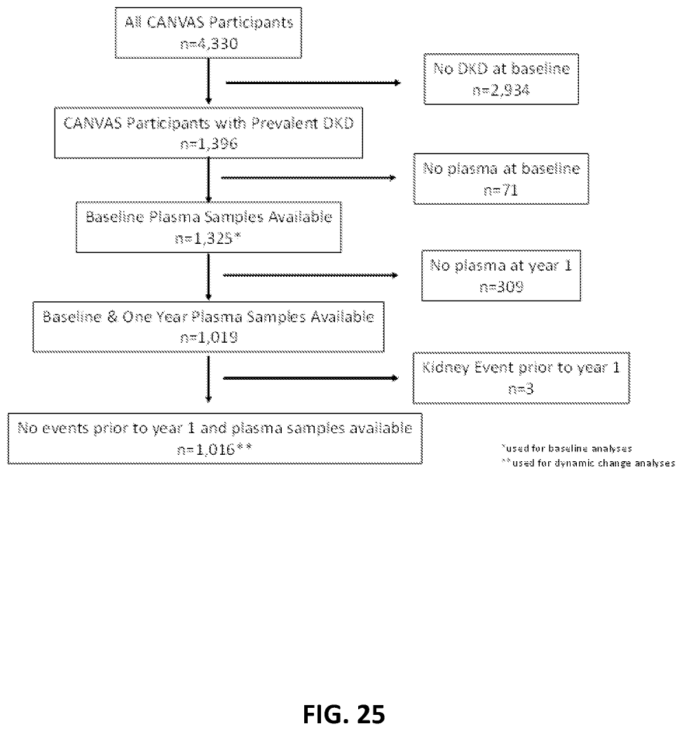

In one example, of 1396 participants in a CANVAS trial with prevalent DKD, 1325 had available blood samples at baseline and 1019 had blood samples available at baseline and at 1 year (as shown in FIG. 25). For the analysis on the association between 1-year changes in biomarkers and subsequent kidney outcome, 3 of the 1016 participants were excluded, since they experienced the time to event kidney outcome events (40% sustained decline or kidney failure) before year 1, leaving 1013 available for the longitudinal analyses. The mean age 64 years, 32% were female, mean eGFR was 65 mL/min/1.73 m2, and the median uACR was 56 mg/g.

Association Between KidneyIntelX with the Composite Kidney Outcome

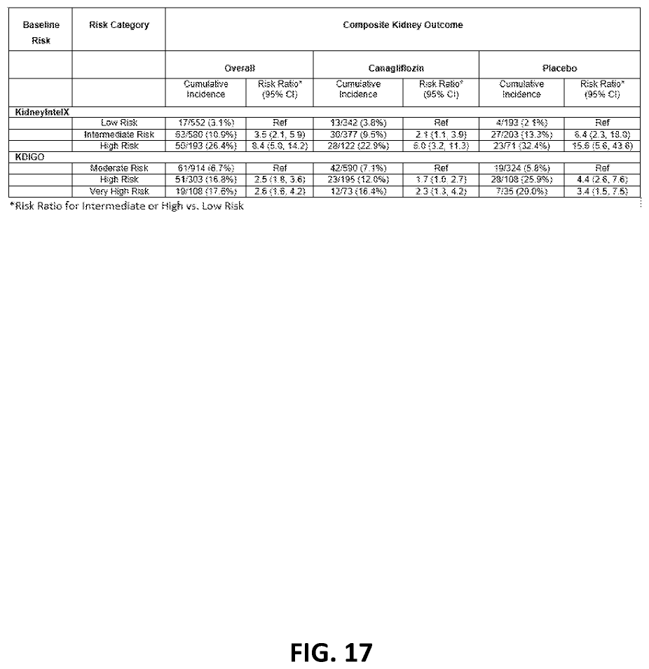

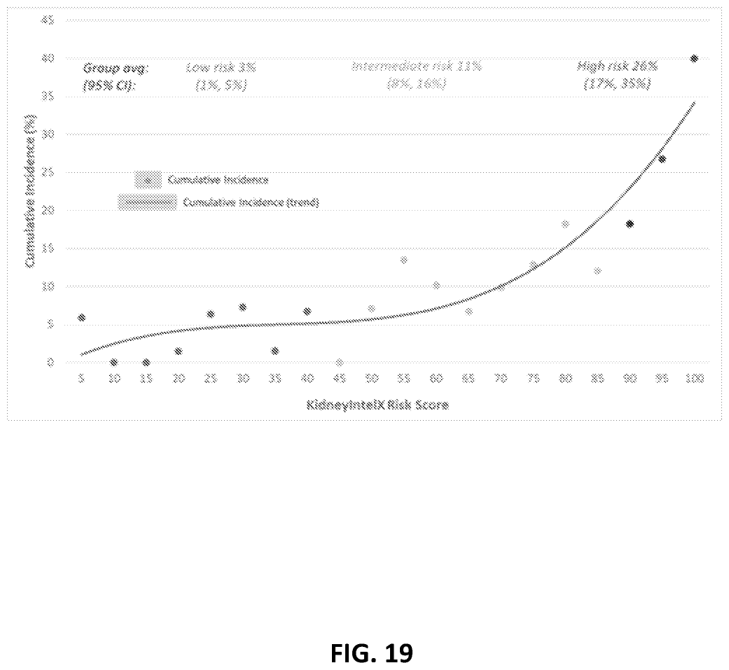

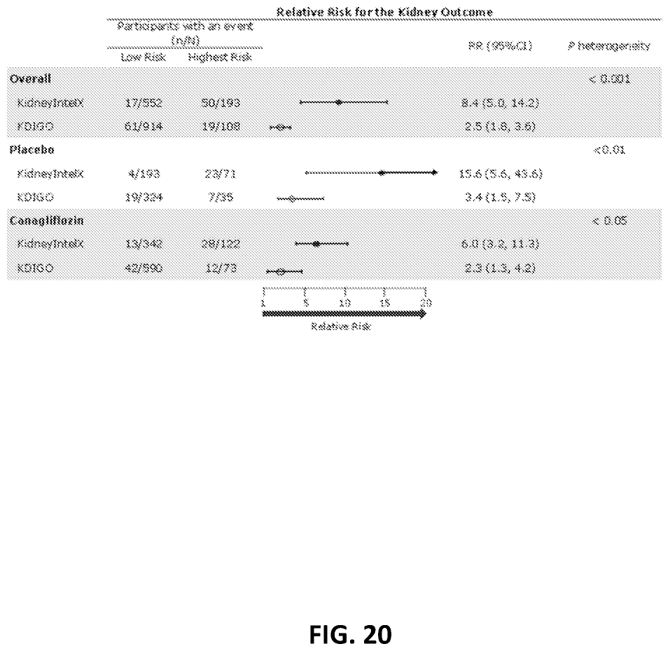

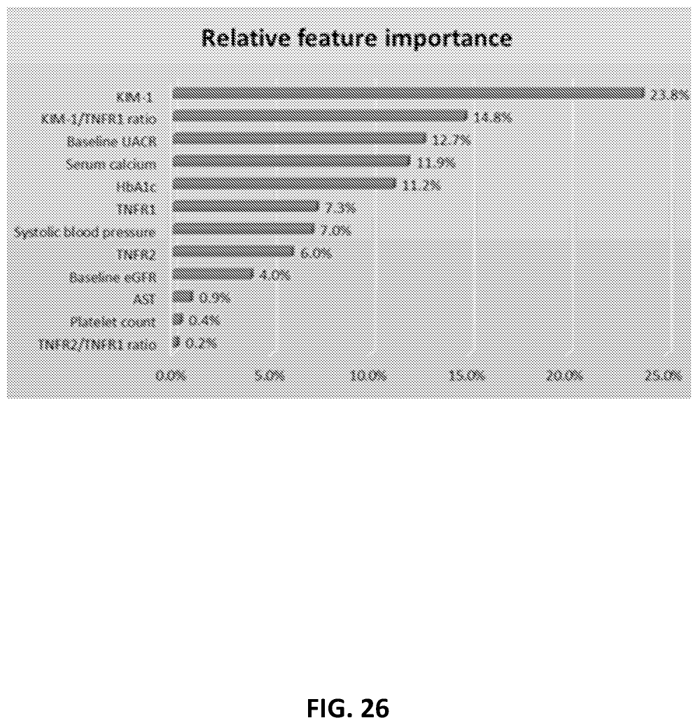

During a mean follow-up of 5.6 years, 131 (9.9%) of the 1325 with baseline DKD experienced the composite kidney outcome. Using risk cutoffs from prior validation studies, the cumulative incidence of the composite kidney outcomes increased as KidneyIntelX algorithm (e.g., performed by diagnosis device 101) risk scores increased (FIG. 19). In terms of the KidneyIntelX algorithm risk strata, 41.7% of participants were classified as low risk, 43.8% were classified as intermediate risk, and 14.6% were classified as high risk. The corresponding incidence of the composite outcome was 3.1%, 10.9%, and 26.4% for low, intermediate and high risk. In the canagliflozin arm, the corresponding incidence was 3.6, 9.5 and 23.0%, yielding a risk ratio of 6.0, 95% CI 3.2-11.3 for the high vs. low risk groups, and in the placebo arm, the corresponding incidence was 2.1, 13.3 and 32.4%, yielding a risk ratio of 15.6, 95% CI 5.6-43.6; FIG. 17 and FIG. 20). By way of illustration for how KidneyIntelX can be incorporated into clinical practice, the risk categorization to the KDIGO categorization based on eGFR and uACR were compared, which stratified 69%, 23% and 8% of the DKD population into “moderately increased risk”, “high risk”, and “very high risk”, with event rates of 7%, 17%, and 18% (relative risk [RR] of 2.3, 95% CI 1.3-4.2 for the “very high risk” vs. “moderately high risk” in the treatment arm and RR of 3.4, 95% CI 1.5-7.5 in the placebo arm). Feature importance is shown in FIG. 26. The net reclassification index for KidneyIntelX vs. KDIGO was 34,2% (22.7% in events and 11.5% in non-events).

Effect of Canagliflozin on Kidney Outcomes (Composite and eGFR Slope) by Baseline KidneyIntelX Risk Strata

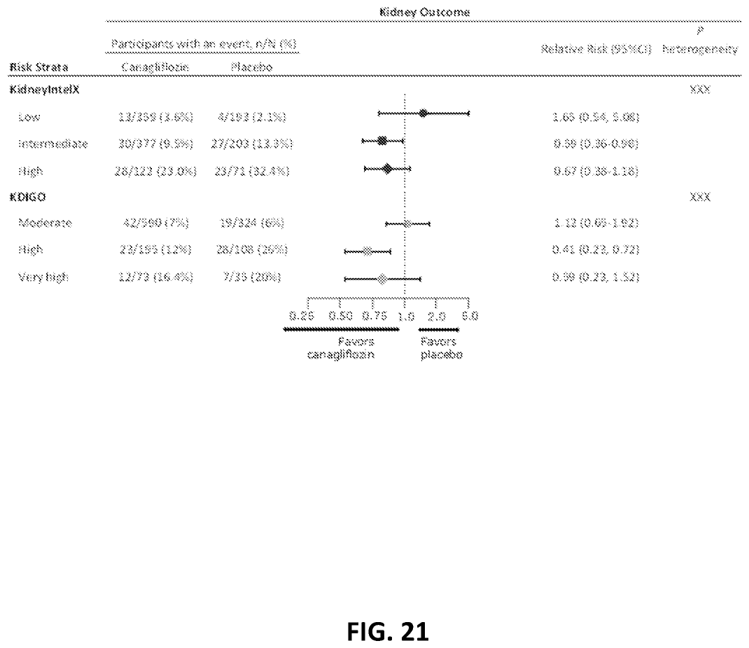

In one example, in all participants with baseline biomarkers, the composite kidney outcome occurred less frequently in the canagliflozin group (8.9%) compared to placebo group (11.6%; RR for cangliflozin vs. placebo 0.70; 95% CI 0.49, 0.99). The treatment effects of canagliflozin vs. placebo varied by baseline strata of KidneyIntelX. In the low-risk stratum, the RR for the kidney outcome was 1.65 (95% CI 0.54-5.08) for canagliflozin vs. placebo, in the intermediate stratum the RR was 0.59 (95% CI 0.36-0.98) and in the high-risk stratum the RR was 0.67 (95% CI 0.38-1.18; FIG. 21). In the high-risk KidneyIntelX stratum, 31.4% experienced the composite kidney outcome in the placebo arm, and 23.5% in the canagliflozin arm (absolute risk reduction (ARR) for canagliflozin of 7.9%).

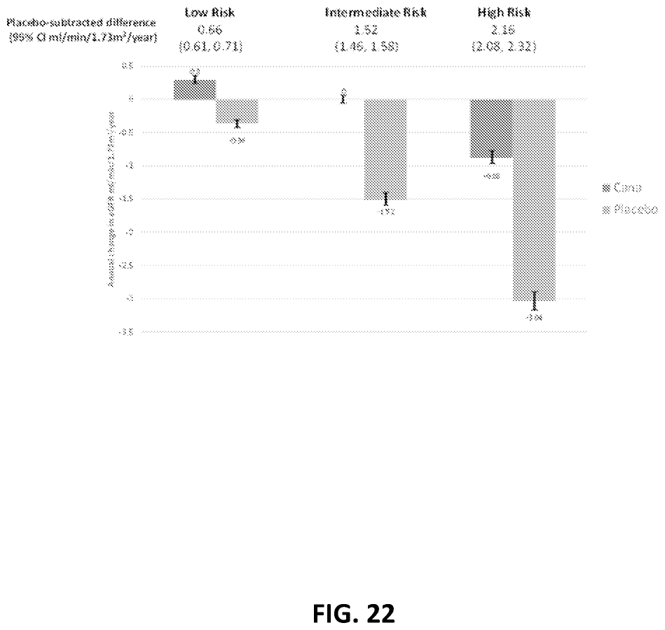

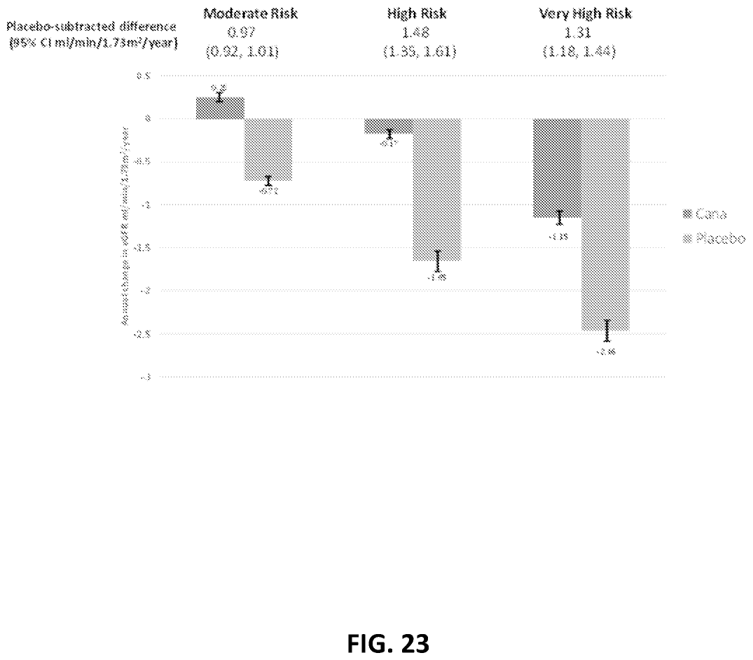

In one example, the effect of canagliflozin vs. placebo on chronic eGFR slopes by KidneyIntelX strata was also examined (FIG. 22 and FIG. 23). There was evidence of greater protection with canagliflozin for eGFR decline in moderate/intermediate risk (1.52 ml/min/1.73 m2) and high risk (2.16 ml/min/1.73 m2) compared to the low risk stratum (0.66 ml/min/1.73 m2). The differences in eGFR slope for canagliflozin vs. placebo in the high risk KidneyIntelX algorithm stratum (2.16 ml/min/1.73 m2) was of greater magnitude when compared to the effect of canagliflozin vs. placebo in the highest risk KDIGO stratum (1.31 ml/min/1.73 m2; p<0.001; as shown in FIG. 22 and FIG. 23).

Effect of Canagliflozin on KidneyIntelX Scores Over Time

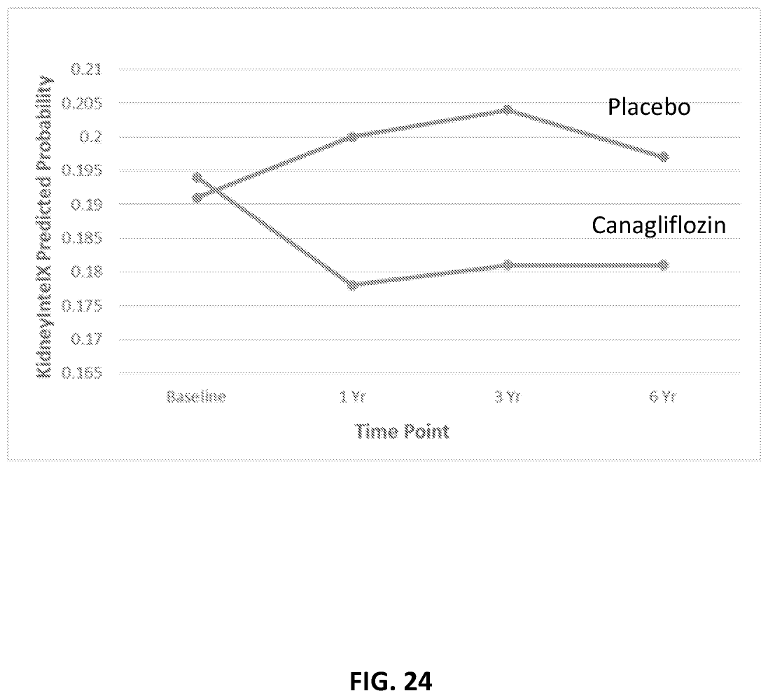

In one example, among the 1066 participants with baseline and year 1 samples available, KidneyIntelX increased from baseline to one year in the placebo group by 6.2% (95% CI 3.8 to 8.6) at year 1and decreased by 5.4% (95% CI −6.9 to −3.9) in those randomized to Canagliflozin (p<0.001). This effect of canagliflozin on KidneyIntelX persisted over time until the end of follow-up (FIG. 24).

Associations Between Changes in KidneyIntelX and Outcomes

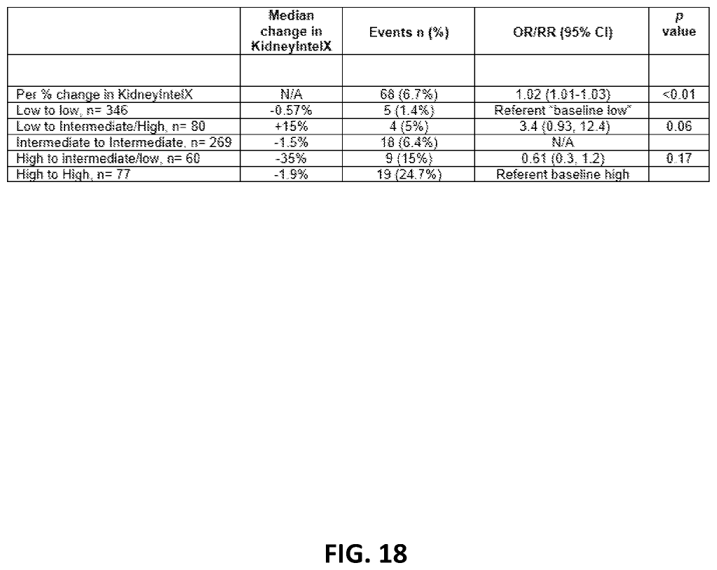

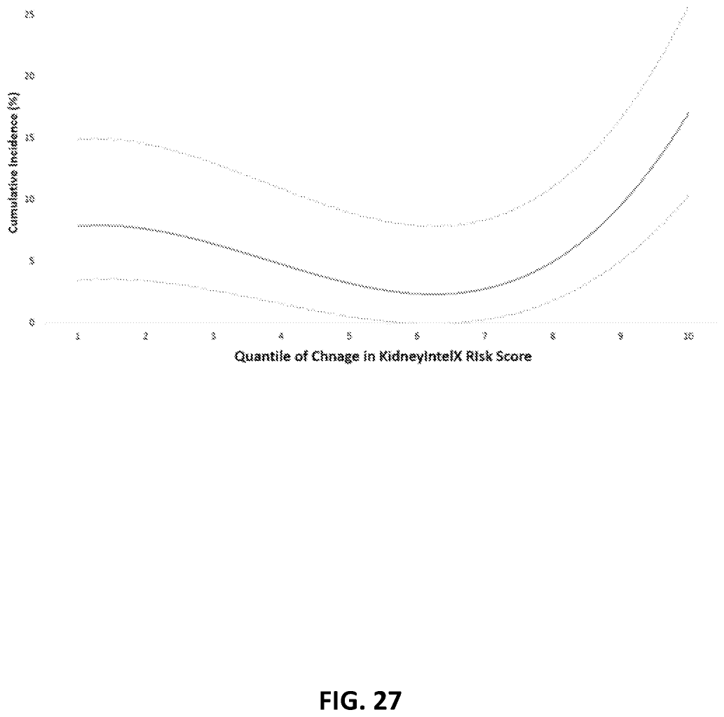

In some instances, an association of a change in KidneyIntelX from baseline to year 1 with the composite kidney outcome among the 1016 participants with available sample and no event prior to year 1 were examined. After adjustment for baseline KidneyIntelX score and randomization arm, each percent reduction in KidneyIntelX was associated with a lower risk of the kidney outcome (adjusted OR 0.98, 95% CI 0.97 to 0.99; p<0.001; as shown in FIG. 27). There was no evidence of interaction by treatment arm (p interaction=0.59). When further grouped by baseline KidneyIntelX risk strata, participants that started as low risk and remained low risk (n=346), only 5 (1.4%) experienced the composite kidney outcome. For those that started low but experienced a change in KidneyIntelX risk group to intermediate or high (n=80), 4 (5%) experienced the event (risk ratio for those changing from low vs. remained low 3.4, 95% CI 0.95-12.6; FIG. 18). In those that started as high risk and remained high risk at year 1 (n=77), 19 individuals (25%) experienced the composite kidney outcome. For those that started high and achieved a reduction to an intermediate or low risk KidneyIntelX risk group (n=60), 9 (15%) experienced the event (risk ratio for those changing from high to intermediate or low vs. those remaining high 0.60, 95% CI 0.3-1.2).

Discussion

KidneyIntelX algorithm is strongly prognostic for future kidney outcomes in patients with type 2 diabetes with existing DKD in cohorts of patients enrolled in broad biobanks linked to real-world electronic medical record (EMR) data. External validation of KidneyIntelX risk stratification performance in a multinational clinical trial population of participants with type 2 diabetes and prevalent DKD were demonstrated in the CANVAS trial. Other potentially beneficial features of risk stratification with KidneyIntelX were also shown. First, effects of canagliflozin on kidney outcomes and eGFR slope were greater in magnitude in those that were scored as high-risk by KidneyIntelX. Second, the SGLT2i canagliflozin decreased KidneyIntelX risk scores over time compared to placebo, and finally, changes in KidneyIntelX at 1 year were associated with subsequent kidney outcomes independent of baseline KidneyIntelX scores. Therefore, KidneyIntelX has clinical utility not only as a prognostic tool, but also has utility for longitudinal assessment and time-updated risk with or without treatment for DKD.

Patients with high KidneyIntelX scores at baseline did derive slightly greater absolute benefit in terms of kidney function decline over time. While decisions to prescribe SGLT2i can be complex and dependent not only on patient-related factors, but also monetary constraints, there is the potential to use KidneyIntelX scoring as a factor to motivate increased utilization by providers and increased acceptance and compliance by patients. KidneyIntelX successfully risk-stratified a large multi-national external cohort for risk of progression of DKD, with larger differences in observed events across KidneyIntelX risk strata compared to KDIGO risk strata and greater differences in eGFR slope for canagliflozin vs. placebo. Canagliflozin treatment reduced KidneyIntelX risk scores over time and changes in the KidneyIntelX score from baseline to 1 year predicted future risk of DKD progression.

Validation of a Machine Learning-Derived Prognostic Test

Methods and apparatus described herein present implementations of accurate models that combine clinical data with soluble tumor necrosis factor receptors (TNFR) 1 and 2 and plasma kidney injury molecule-1 (KIM-1) biomarkers to predict progression of kidney disease.

Widespread electronic health record (EHR) usage provides the potential to leverage thousands of clinical features. Known statistical approaches are inadequate to leverage this data due to feature volume, unaligned nature of data, and correlation structure. Methods and apparatus described herein use plasma samples linked to longitudinal clinical data to examine an ability of a prognostic test (e.g., KidneyIntelX) that uses machine learning algorithms to predict progressive decline in kidney function and kidney outcomes in two discrete, high-risk patient populations, type 2 diabetes (T2D) and APOL1-HR genotype.

Data: BioMe Biobank at Icahn School of Medicine at Mount Sinai (ISMMS)

The BioMe Biobank at ISMMS is an institutional review board—approved biorepository that includes consented access to the patients' EHR from a diverse community in New York City, N.Y. Operations were initiated in 2007 and include direct recruitment from more than 30 broadly selected clinical sites. Two subpopulations: (1) T2D, enrollment eGFR 45-90 ml/min, and ≥3 years of follow-up data; and (2) APOL1-HR, enrollment eGFR >30 ml/min and ≥3 years of follow-up data, were selected (number of individuals in the data is 1369).

Ascertainment and Definition of the Kidney End Point

In some instances, eGFR using the CKD—Epidemiology Collaboration equation and eGFR slope using a minimum of three values from baseline were determined (e.g., using the diagnosis device 101 as described with respect to FIG. 1). The primary composite outcome was comprised of three components: progressive decline in kidney function, defined as an eGFR slope decline of ≥5 ml/min per 1.73 m2 per year, or a sustained (confirmed ≥3 months later) decline in eGFR of >40% from baseline, or “kidney failure” defined by sustained eGFR <15 ml/min per 1.73 m2 confirmed at least 30 days later or long-term maintenance dialysis or kidney transplant (i.e., ESKD).

Ascertainment of Clinical Variables in BioMe Biobank

In some instances, demographic-related factors such as, for example, sex and race can be obtained from an enrollment questionnaire. Clinical data can be extracted for all continuous variables at the time of and before baseline from the EHR with concurrent time stamps. Hypertension and T2D can be determined using phenotyping algorithms. Cardiovascular disease and heart failure can be determined by a validated algorithm and International Classification of Diseases, Ninth/Tenth Revision codes, respectively. A participant is considered to be on an angiotensin-converting enzyme inhibitor or angiotensin receptor blocker if they had a concurrent prescription at enrollment. Thereafter, follow-up time can be calculated from enrollment to the latest visit. In some instances, only variables present in a pre-set percentage (e.g., above >70%) of subjects (except UACR/BP due to their established clinical importance) can be included and used for training of the KidneyIntelX algorithm.

Biospecimen Storage and Analyte Measurement

In some instances, plasma specimens collected on the day of BioMe enrollment can be stored continuously at −80° C. The biomarkers can be measured in a multiplex format using the Meso Scale platform (e.g., Meso Scale Diagnostics, Gaithersburg, Md.), employing proprietary electrochemiluminescence detection methods combined with patterned arrays allowing for analyte multiplexing. The intra- and interassay coefficient of variation for quality control samples with known low, moderate, and high concentrations of each biomarker run on each plate can be 3.5%, 3.9%, and 4.5%; and 12.4%, 10.8%, and 7.7% for TNFR1, TNFR2, and KIM-1, respectively. In some instances, laboratory personnel can be blinded to clinical information.

Statistical Analyses

In some instances, descriptive results for the participants' baseline characteristics and biomarkers via means and standard deviations (SDs) or, for skewed variables, medians and interquartile ranges (IQRs) can be expressed.

In some instances, a random forest model can be used with two sets of input data: (1) biomarker concentrations/ratios; and (2) EHR features including laboratory values, diagnosis/procedure codes, demographics (e.g., age, sex, race, etc.), medications, healthcare encounter history, and/or the like. Missing UACR values can be imputed to 10 mg/g, missing blood pressure (BP) values can be imputed based on multiple predictors (age, sex, race, and antihypertensive medications), and median value imputation can be used for other missing values. As a result, meta-features can be created from variables including maximum, minimum, median, variability, and change over time to account for longitudinal aspect and repetitive nature of the variables. For model development, the two sets of input data (e.g., clinical data) can be randomly and demographically split to create an 80%:20% training and test set, respectively, with 10-fold cross-validation on all candidate models.

Further iterations of the random forest model can be performed by tuning three hyperparameters. In some instances, hyperparameter 1 can be a number of decision trees, hyperparameter 2 can be a number of variables randomly selected for splitting at each node, and hyperparameter 3 can be a minimum size of terminal nodes. The random forest model with hyperparameters resulting in the best area under the receiver operator characteristic curve (AUC) can be chosen.

In some instances, risk probabilities for a composite kidney end point can be generated using the final model on all subjects from both cohorts (T2D and APOL1-HR) and then scaled to generate a continuous score. Performance of the KidneyIntelX can be compared to a validated clinical model that includes a regression equation for 40% eGFR decline prediction including age, sex, race, eGFR, cardiovascular disease, smoking, hypertension, body mass index, and UACR in individuals who are nondiabetics and the aforementioned variables plus insulin, diabetes medications, and hemoglobin Alc for patients with T2D. In some instances, all differences between AUCs using the DeLong test for comparisons can be used.

In some instances, thresholds of the risk score to define low-, intermediate-, and high-risk strata in each cohort can be examined. The low-risk stratum can be set to encompass 50% of the study population, and the thresholds for the high-risk stratum can be assessed to classify the top 10%, 15%, and 20% highest risk in each cohort. The remaining population can be defined as the intermediate-risk stratum. Therefore, sensitivity, specificity, and positive predicted values/negative predicted values (PPV/NPV) for the high-risk and low-risk cutoffs can be calculated and compared these to the validated clinical model. The goodness-of-fit statistics (e.g., Hosmer-Lemeshow) can be used to assess calibration.

In some instances, subgroup/sensitivity analyses can be conducted in individuals with existing CKD (eGFR <60 ml/min per 1.73 m2 and/or UACR >30 mg/g at baseline), and using only data from ≤1 year before biomarker measurement (i.e., “contemporary data,” to ensure that the KidneyIntelX was robust in advanced stages of the disease and performed equally well with clinical data limited to a year before biomarker measurement). In addition, a trained and tested random forest model that did not include any of the biomarkers (TNFR1, TNFR2, or KIM-1) in both cohorts can be generated. Therefore, a performance of the full KidneyIntelX model for the individual components of the composite kidney end point can be examined. For that purpose, a Kaplan-Meier survival analyses for time-dependent outcomes of 40% decline and kidney failure with hazard ratios was conducted using the Cox proportional hazards method for the high-risk (top 15%) versus the intermediate- and low-risk strata (bottom 50%).

Results: Baseline Characteristics of Cohorts, Patients with T2D (n=871)

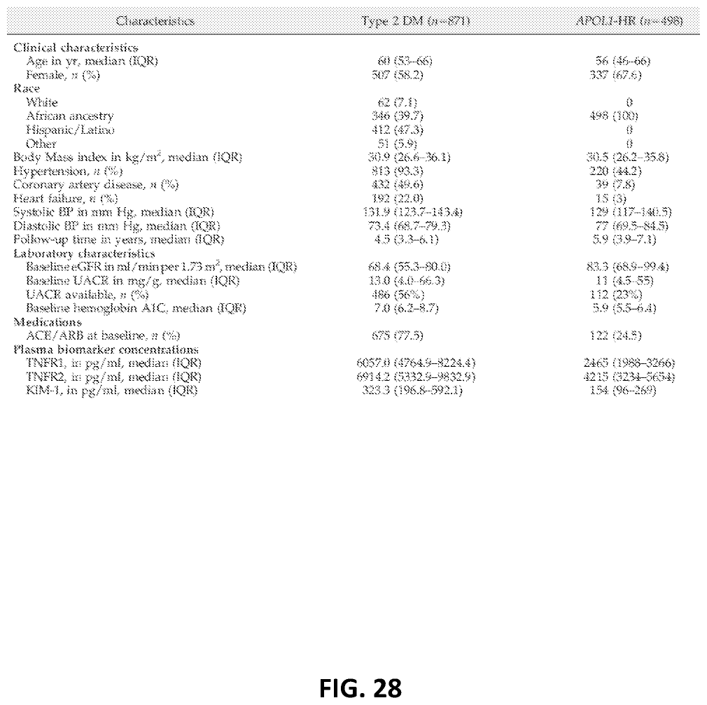

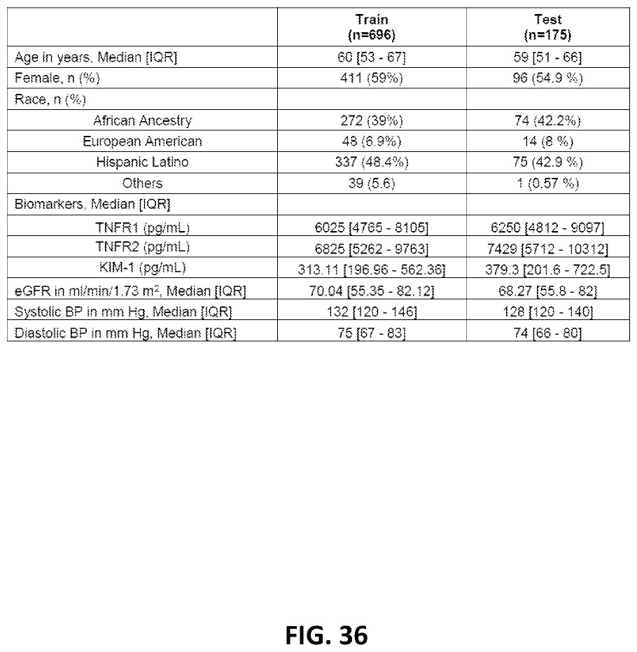

As shown in FIG. 28 and FIG. 36, a median age of the cohorts was 60 years, 507 (58%) were female, and the median eGFR was 68 ml/min per 1.73 m2. The most common comorbidities were hypertension (93%), coronary heart disease (50%), and heart failure (22%). The majority (77%) were on angiotensin-converting enzyme inhibitors or angiotensin receptor blockers. Patient characteristics including events between the training and test cohorts were balanced.

Results: Baseline Characteristics of Cohorts, Patients with APOL1-HR (n=498)

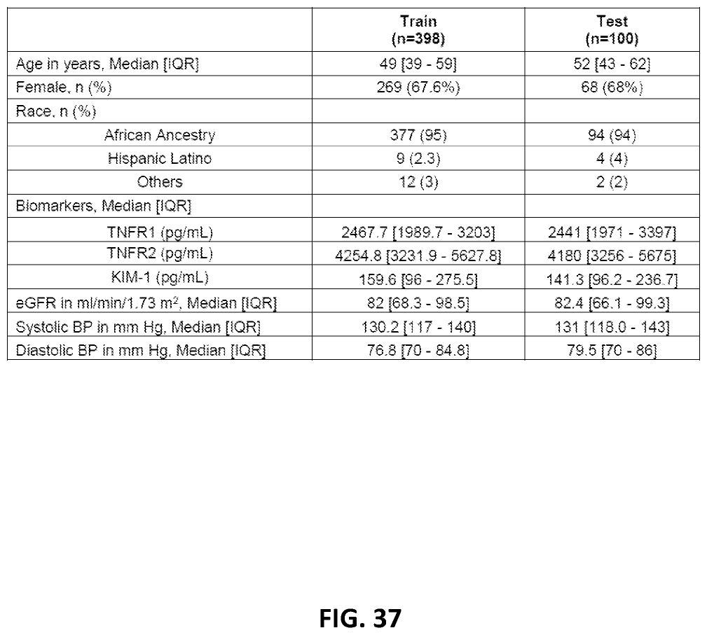

As shown in FIG. 28. and FIG. 37, the median age was 56 years, 337 (67.6%) were female, and the median eGFR was 83.3 ml/min per 1.73 m². The prevalence of comorbidities were lower than the T2D cohort: hypertension (44%), coronary heart disease (8%), and heart failure (3%). Patient characteristics including events between the training and test cohorts were comparable.

Results: Composite Kidney End Point

For participants with T2D, 201 of the 871 (23%) experienced the composite kidney end point over a median follow-up of 4.6 (IQR, 3.4-5.6) years. In participants with the APOL1-HR genotypes, 90 of the 498 (18%) experienced the composite kidney end point over a median follow-up of 5.9 (IQR, 3.9-7.1) years.

Results: Machine Learning (e.g., Random Forest) Model for Prediction of the Composite Kidney End Point

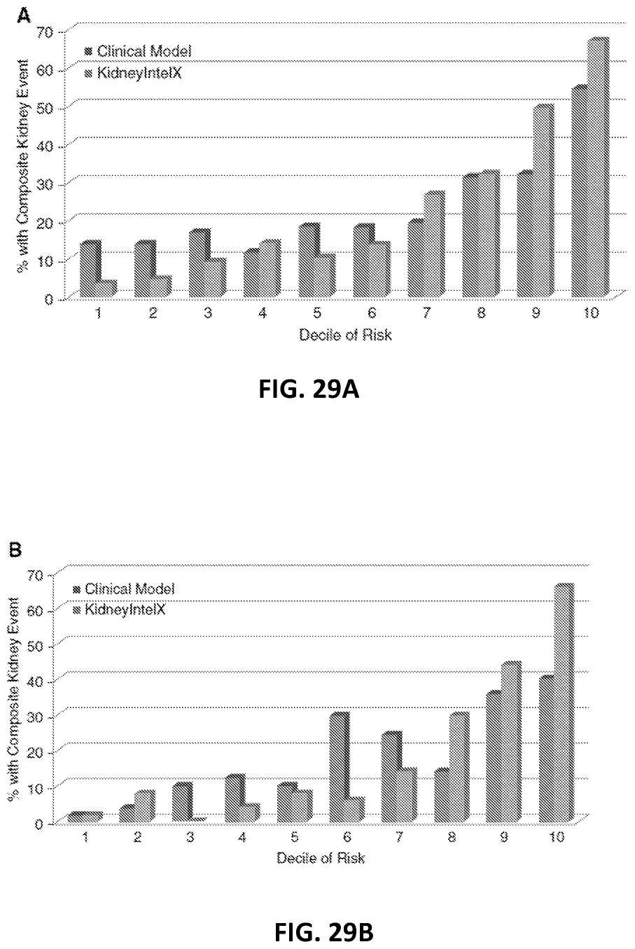

Observed composite kidney event by deciles of risk with KidneyIntelX versus the standard clinical model are shown in FIGS. 29A and 29B. For patients with T2D, applying 10-fold cross-validation, the KidneyIntelX AUC in the training set (80%, n=697) for the composite kidney end point was 0.81 (95% CI, 0.80 to 0.82) and 0.77 (95% CI, 0.75 to 0.79) in the test set (20%, n=174). By comparison, the clinical model had an AUC of 0.66 (95% CI, 0.65 to 0.67) in the entire T2D cohort (n=871).

For patients with APOL1-HR genotypes, applying 10-fold cross-validation, the AUC for KidneyIntelX in the training set (80%, n=398) was 0.86 (95% CI, 0.84 to 0.87) and 0.80 (95% CI, 0.77 to 0.83) in the test set (20%, n=99). The clinical model (9) had an AUC of 0.72 (95% CI, 0.71 to 0.73) in the APOL1-HR cohort (n=498).

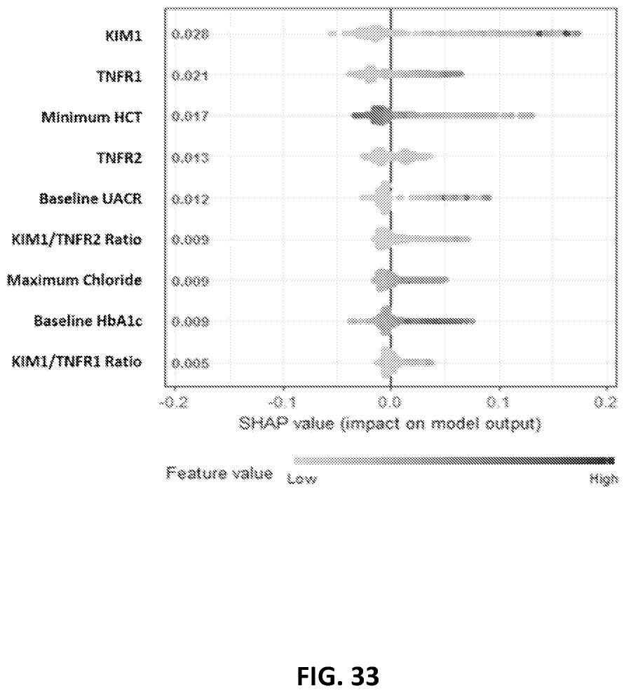

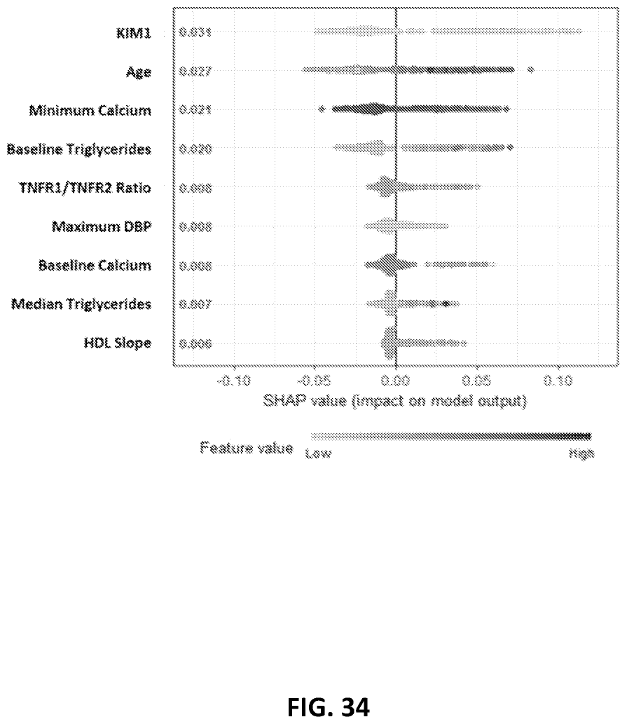

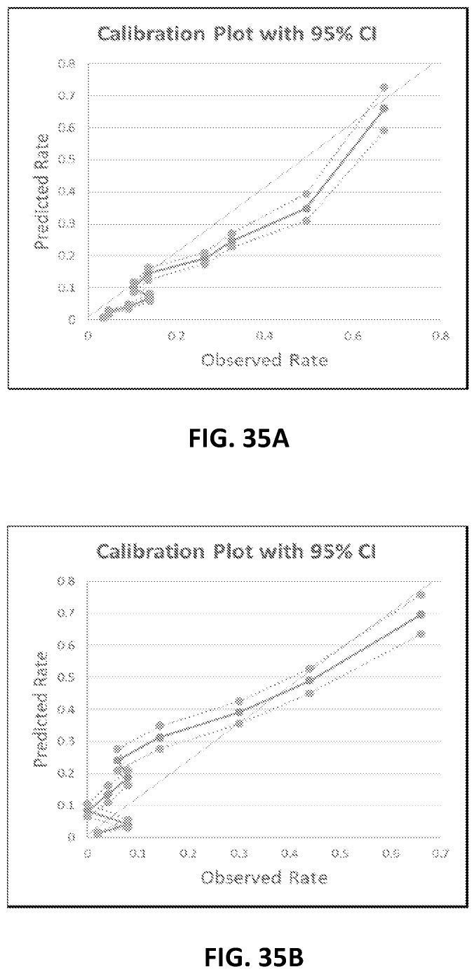

In both the T2D and APOL1-HR cohorts, the features noted to contribute most to performance were the three plasma biomarkers (TNFR1, TNFR2, and KIM-1) or their ratios of individual biomarker values to each other (i.e., three ratios) and laboratory values or vital signs (either baseline or changes over time) that are linked to kidney disease (as shown in FIG. 33 and FIG. 34). The P values of the Hosmer-Lemeshow goodness-of-fit test for the prognostic models were 0.15 and 0.11, indicating there was no significant difference between the predicted and observed outcomes (as shown in FIGS. 35A and 35B).

Results: KidneyIntelX Cutoffs for the Composite Kidney End Point (Entire T2D [n=871] and APOLI-HR [n=498] Cohorts

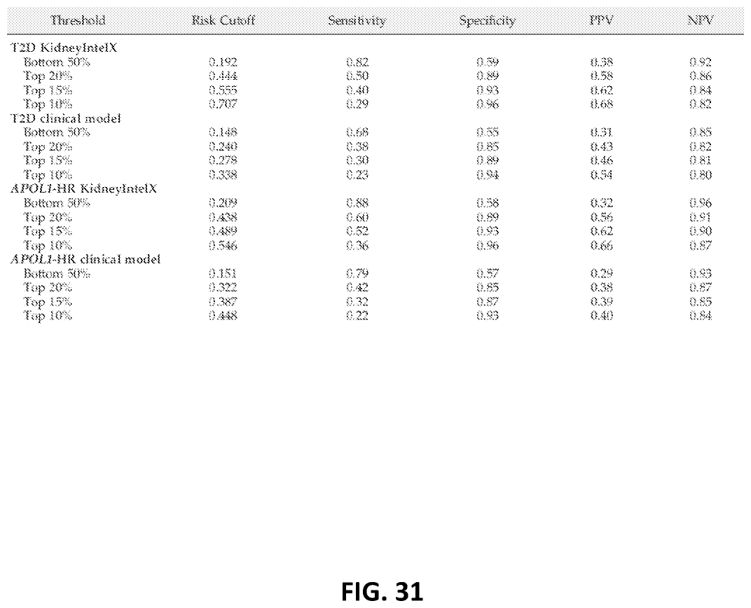

The PPVs of KidneyIntelX were 58%, 62%, and 68% in the top 20%, 15%, and 10% highest risk of the T2D population versus 43%, 46%, and 54% in the top 20%, 15%, and 10% of highest risk as classified by the clinical model (P<0.01 for all comparisons; as shown in FIG. 31). The PPVs of KidneyIntelX were 56%, 62%, and 66% in the top 20%, 15%, and 10% highest risk of APOL1-HR population versus PPV of 38%, 39%, and 40% of the highest risk as classified by the clinical model (P<0.01 for all comparisons). When applying cutoffs for the lowest 50% of risk in the T2D cohort, the NPV for KidneyIntelX compared with the clinical model was 92% versus 85% (P=0.76). Similarly, for the APOL1-HR cohort, the NPV for KidneyIntelX compared with the clinical model was 96% versus 93% (P=0.93).

Results: Supplementary and Sensitivity Analyses, Prevalent CKD

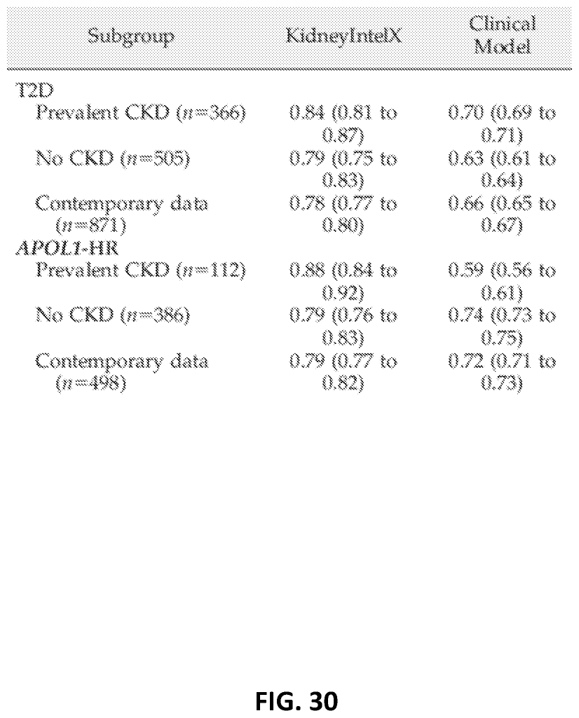

When a performance of KidneyIntelX was stratified by baseline CKD (i.e., eGFR ≤60 ml/min per 1.73 m2 and/or UACR ≥30 mg/g at baseline, n=366), 27.6% experienced the primary composite kidney end point during follow-up, compared with 12.5% in those without baseline CKD (n=505). The AUC was 0.84 (95% CI, 0.81 to 0.87) in individuals with prevalent CKD versus 0.79 (95% CI, 0.75 to 0.83) in those without CKD (FIG. 30). For APOL1-HR individuals, 112 had baseline prevalent CKD, of which 31.2% experienced the composite kidney end point. In this subgroup, the KidneyIntelX model produced an AUC of 0.88 (95% CI, 0.84 to 0.92) versus the 386 without baseline CKD, the AUC was 0.79 (95% CI, 0.77 to 0.82; as shown in FIG. 30).

Results: Supplementary and Sensitivity Analyses, Contemporary Data

Using contemporary data only (data within one year before enrollment and biomarker measurement), the discriminatory performance of the KidneyIntelX model in both the T2D (AUC, 0.78; 95% CI, 0.77 to 0.80) and APOL1-HR (AUC, 0.79; 95% CI, 0.77 to 0.82) cohorts were similar when all clinical data were available, demonstrating KidneyIntelX is not dependent on multiyear history to provide accurate prognostic information (as shown in FIG. 30).

Results: Supplementary and Sensitivity Analyses, Random Forest Model with and without Biomarkers

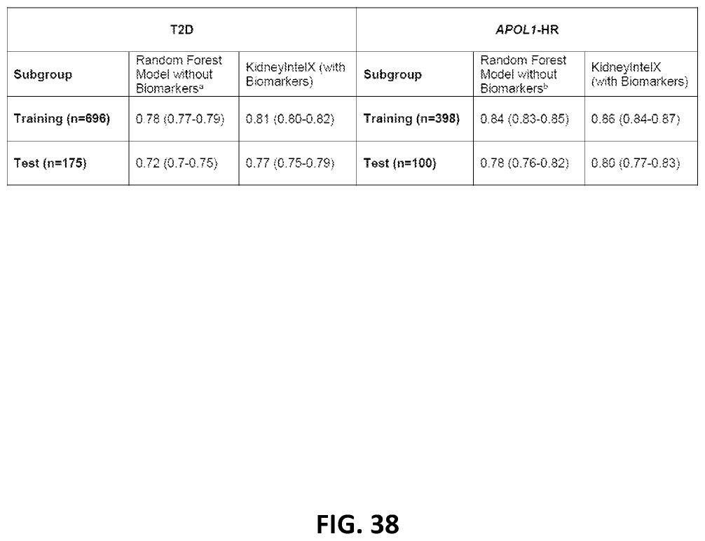

A newly created random forest model with different clinical features that did not include any of the biomarkers (TNFR1, TNFR2, or KIM-1) in both cohorts had lower training and test AUCs than the full KidneyIntelX model with plasma biomarkers and their ratios (as shown in FIG. 38).

Results: Supplementary and Sensitivity Analyses, Discrimination for Individual Components of the Composite Kidney End Point

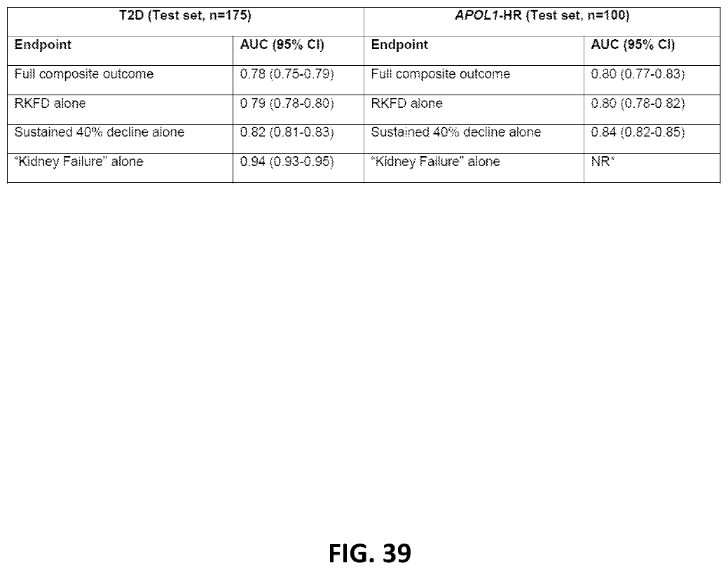

The discriminatory performance of KidneyIntelX (trained for the entire composite end point) for the individual components of the composite end point (RKFD alone, sustained 40% decline alone, or kidney failure alone) in the test cohorts for T2D and APOL1-HR was comparable (as shown in FIG. 39).

Results: Time-to-Event Analyses for 40% Sustained Decline in eGFR or Kidney Failure

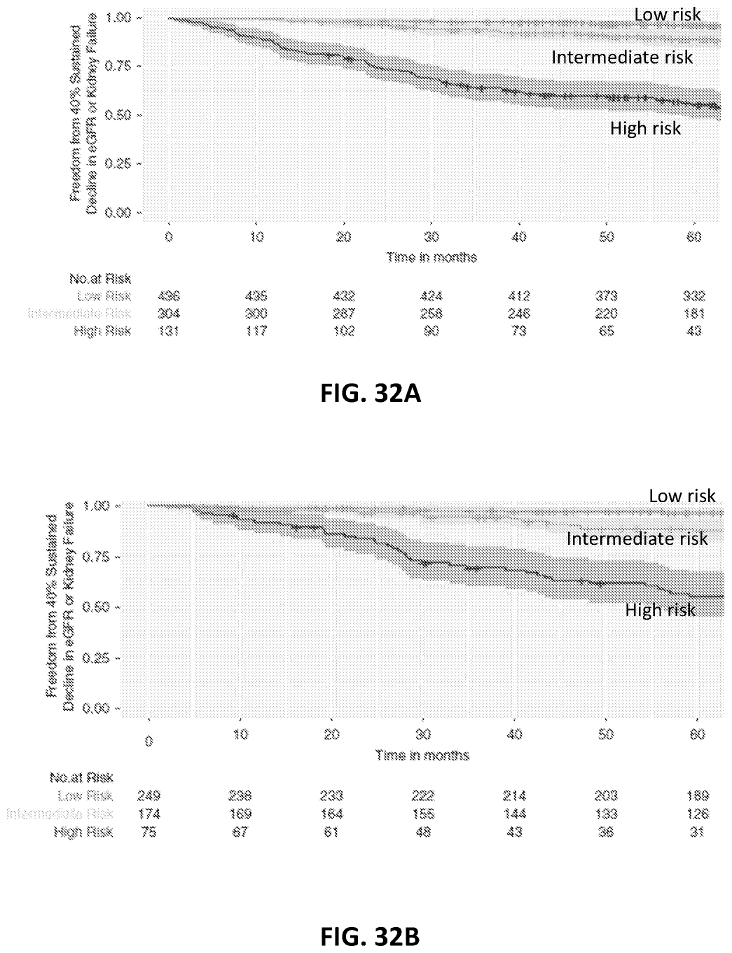

Patients with high-risk KidneyIntelX scores (top 15% in T2D and in APOL1-HR) had a greater risk of progression to time-to-event categoric outcomes of 40% sustained decline or kidney failure than patients in the low- or medium-risk strata combined (hazard ratios 9.9 [95% CI, 6.7to 14.6] and 9.1 [95% CI, 5.8 to 14.3]), respectively. Separation of the high-risk stratum Kaplan-Meier curve occurred within the first year and progressively declined over time (FIGS. 32A and 32B).

Results

Using two large cohorts (T2D and APOL1-HR) of patients at high risk for progressive kidney function decline, with banked plasma samples linked to the corresponding EHR data, a prognostic model combining EHR data and three plasma biomarkers to predict a composite kidney end point was developed. The composite kidney end point included RKFD (decline of ≥5 ml/min/1.73 m2/year), 40% sustained decline, or kidney failure. The KidneyIntelX prognostic model was more accurate for predicting the risk of kidney function decline than validated clinical models. The ability to identify a distinct patient group with the composite kidney end point with a PPV of >55% allows for more appropriate future patient management including nephrologist referral, improved awareness of kidney health, and guidance toward more targeted, intensive therapies to slow progression. The demonstrated PPV in the high-risk stratum represents a three-fold improvement over the observed baseline event rate in the two populations.

In practice, the prediction of kidney disease progression in patients with T2D and/or APOL1-HR is challenging, particularly in patients with largely preserved kidney function. There are two major problems contributing to the difficulties in early identification and prediction: (1) serum creatinine/eGFR and UACR are relatively insensitive and nonspecific biomarkers, with significant fluctuations and variability in early stages of CKD; and (2) the prevalent standard includes recursive scores incorporating only a single (baseline) value of a selected predictive feature and does not include longitudinal data.

Aforementioned results demonstrate that analyzing these biomarkers (soluble TNFR1, soluble TNFR2, and plasma KIM-1) with clinical information using machine learning model(s) and/or techniques presented herein can significantly improve the discrimination/prediction of composite kidney end points. Biomarkers that can be measured during a routine clinical encounter can be combined with longitudinal EHR data present in most healthcare systems for optimal prediction.

Aforementioned integrated approach has near-term clinical implications, especially when linked to clinical decision support and embedded care pathways within the EHR. For example, patients with a high KidneyIntelX risk score, with a probability of >50% for the kidney end point, should be referred to a nephrologist, which has been associated with improved outcomes. In addition, referral of high-risk patients to a dietician and the delivery of educational materials regarding the importance and consequences of CKD should increase awareness and facilitate motivation for changes in lifestyles and behavior. Finally, the optimization of medical therapy including renin-angiotensin-aldosterone system inhibitors, statins for cardiovascular risk management, and intensification of antihypertensive medication to meet guideline-recommended BP targets can be pursued. The application of sodium glucose transporter-2 inhibitors might also be advantageous in the high KidneyIntelX score group with T2D given recent data on robust renoprotection.

Alternatively, patients with a low-risk score could be clinically managed by their primary care provider and have a standard-of-care treatment with scheduled monitoring of their KidneyIntelX results. Finally, patients with an intermediate-risk score would be recommended for the standard of care and retesting longitudinally. Such patients may demonstrate changes in KidneyIntelX results based on behavioral changes, clinical parameters, and treatment adjustments over time, with appropriate clinical actions as necessary. The overall approaches presented herein would not only benefit individual patient outcomes but also positively affect health systems where there is uncertainty about which patients to refer to a limited number of subspecialists.

Some embodiments described herein relate to methods. It should be understood that such methods can be computer implemented methods (e.g., instructions stored in memory and executed on processors). Where methods described above indicate certain events occurring in certain order, the ordering of certain events can be modified. Additionally, certain of the events can be performed repeatedly, concurrently in a parallel process when possible, as well as performed sequentially as described above. Furthermore, certain embodiments can omit one or more described events.

Examples of computer code include, but are not limited to, micro-code or micro-instructions, machine instructions, such as produced by a compiler, code used to produce a web service, and files containing higher-level instructions that are executed by a computer using an interpreter. For example, embodiments can be implemented using Python, Java, JavaScript, C++, and/or other programming languages, packages, and software development tools.

The drawings primarily are for illustrative purposes and are not intended to limit the scope of the subject matter described herein. The drawings are not necessarily to scale; in some instances, various aspects of the subject matter disclosed herein can be shown exaggerated or enlarged in the drawings to facilitate an understanding of different features. In the drawings, like reference characters generally refer to like features (e.g., functionally similar and/or structurally similar elements).

While various inventive embodiments have been described and illustrated herein, those of ordinary skill in the art will readily envision a variety of other steps, means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, steps, biomarkers, blood components/metabolites, materials, functionality and configurations described herein are meant to be an example and that the actual parameters, steps, biomarkers, blood components/metabolites, materials, functionality and configurations will depend upon the specific application/use or applications/uses for which the inventive teachings is/are used. Those skilled in the art will recognize or be able to ascertain using no more than routine experimentation, many equivalents to the specific inventive embodiments described herein. It is, therefore, to be understood that the foregoing embodiments are presented by way of example only and that, within the scope of the appended claims, equivalents thereto, and any claims supported by the present disclosure, inventive embodiments may be practiced otherwise than as specifically described and claimed. Inventive embodiments of the present disclosure are directed to each individual feature, system, article, material, kit, method, functionality, and step, described herein. In addition, any combination of two or more such features, systems, articles, materials, kits, methods, and steps, if such features, systems, articles, materials, kits, methods, and steps, are not mutually inconsistent, is included within the inventive scope of the present disclosure. Embodiments disclosed herein may also be combined with one or more features, as well as complete systems, devices and/or methods, to yield yet other embodiments and inventions. Moreover, some embodiments, may be distinguishable from the prior art by specifically lacking one and/or another feature disclosed in the particular prior art reference(s); i.e., claims to some embodiments may be distinguishable from the prior art by including one or more negative limitations.

Various inventive concepts are embodied as one or more methods, of which at least one example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

and all references to publications or other documents, including but not limited to, patents, patent applications, articles, webpages, books, etc., presented anywhere in the present application, are herein incorporated by reference in their entirety. Moreover, all definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

What is claimed is:

1. A method, comprising:
receiving, for each respective human training subject in a plurality of diabetic human training subjects:
a respective set of biomarker data from a biological sample collected from the respective human training subject at a respective first time;
a respective first set of human subject data (HSD) collected from the respective human training subject at the respective first time; and
a respective second set of HSD collected from the respective human training subject at a respective second time after the respective first time;
determining, for each respective human training subject in the plurality of diabetic human training subjects, a respective indication of whether the respective human training subject experienced progressive decline in kidney function based on at least the respective second set of HSD;
training a machine learning model against, for each respective human training subject in the plurality of diabetic human training subjects (a) a plurality of features derived from at least the respective set of biomarker data collected at the respective first time and the respective first set HSD collected at the respective first time, and (b) a respective indication of whether the respective human training subject experienced progressive decline in kidney function;
receiving a set of biomarker data and a first set of HSD, for a diabetic human test subject not included in the plurality of diabetic human training subjects, collected at a first time for the diabetic human test subject; and
executing, after the training, the machine learning model to generate an indication of whether the diabetic human test subject will experience progressive decline in kidney function over the period of time, based on the set of biomarker data and the first set of HSD for the diabetic human test subject.

2. The method of claim 1, wherein the machine learning model determines a relationship between (i) a plurality of features derived from at least the set of biomarker data and the first set of HSD and (ii) an indication of whether a diabetic human will experience progressive decline in kidney function over a period of time.

3. The method of claim 1, further comprising:
receiving, for each respective human training subject in the plurality of diabetic human training subjects, a respective third set of HSD collected from the respective human training subject at a third time before the first time.

4. The method of claim 1, further comprising:
receiving, for each respective human training subject in the plurality of diabetic human training subjects, a respective fourth set of HSD collected from the respective human training subject at a third time after the second time.

5. The method of claim 1, wherein a subset of the plurality of diabetic human subjects has chronic-kidney-disease (CKD).

6. The method of claim 1, wherein the biomarker data of the plurality of diabetic human subjects indicates a level of at least one of the following biomarkers: sTNFR-1, sTNFR-2, KIM-1, and ratios to one another of any of the preceding.

7. The method of claim 6, further comprising:
detecting the biomarker data of the diabetic human subject in a biological sample of the diabetic human subject.

8. The method of any of claims 1-7, further comprising:
obtaining the first set of HSD or the second set of HSD of the diabetic human subject, the first set of HSD or the second set of HSD including a metabolic factor, a health-related factor, or a demographic-related factor.

9. The method of claim 8, wherein the metabolic factor includes at least one of a Serum Albumin level, a Serum Calcium level, liver enzymes (AST) level, or a Platelet Count.

10. The method of claim 8, wherein the metabolic factor includes at least one of a Hemoglobin-AlC (HbAlC) level, a Urine Albumin-Creatinine Ratio (UACR), a low density lipoprotein cholesterol level, a high density lipoprotein cholesterol level, a triglyceride level, a systolic blood pressure value, a Glomerular Filtration Rate, or a diastolic blood pressure value.

11. The method of claim 8, wherein the health-related factor includes a body-mass-index (BMI) value, a status of past smoking, or a status of current smoking.

12. The method of claim 8, wherein the first set of HSD or the second set of HSD include at least one of a Serum Calcium level, an AST level, a Platelet Count, Hemoglobin-AlC (HbAlC) level, a Urine Albumin-Creatinine Ratio (UACR), a systolic blood pressure value, or a Glomerular Filtration Rate.

13. The method of claim 8, wherein the demographic-related factor includes age, gender, ethnicity, income, education, or employment history.

14. The method of claim 1, wherein the period of time less than 5 years.

15. The method of any of claims 1-14, wherein the biomarker data of the plurality of diabetic human subjects and the first set of HSD of the plurality of diabetic human subjects are split into derivation data and validation data.

16. The method of any of claims 1-15, wherein the plurality of diabetic human subjects are from a first population from a first geographical location and a second population from a second geographical location.

17. The method of any of claims 1-15, wherein the plurality of diabetic human subjects are from a first population from a first healthcare setting and a second population from a second healthcare setting.

18. The method of claim 15, wherein the derivation data are split into training data and test data.

19. The method of claim 15, further comprising:
executing, after the training, the machine learning model based on the validation data.

20. The method of any of claims 1-19, wherein the machine learning model includes a random forest model, deep learning model, a least absolute shrinkage and selection operator (LASSO) model, an eXtreme Gradient Boosting (XGBoost) model, or a support vector machine (SVM).

21. The method of claim 1, further comprising:
performing, during training the machine learning model, a multi-fold cross-validation.

22. The method of claim 1, further comprising:
classifying, before training the machine learning model, the first set of HSD or the second set of HSD of the plurality of diabetic human subjects into a plurality of non-overlapping categories.

23. The method of claim 1, wherein the first set of HSD or the second set of HSD of the plurality of diabetic human samples include Related Health Problems (ICD) codes or Current Procedures Terminology (CPT) codes, each ICD code from the ICD codes or each CPT code from the CPT codes are associated with a Boolean variable and a timestamp.

24. The method of any of claims 1-23, wherein the first set of HSD or the second set of HSD of the plurality of diabetic human subjects include medication data and laboratory values for each diabetic human subject from the plurality of diabetic human subjects.

25. The method of claim 24, further comprising:
mapping, before training the machine learning model, the medication data to RxNorm codes, the training the machine learning model including training the machine learning model based on the RxNorm codes.

26. The method of claim 24, further comprising:
mapping, before training the machine learning model, the laboratory values to Logical Observation Identifiers Names and Codes (LOINC) code, the training the machine learning model including training the machine learning model based on the LOINC codes.

27. The method of claims 1, wherein the machine learning model is configured to predict a composite kidney endpoint of progressive decline in kidney function.

28. The method of claim 1, wherein progressive decline in kidney function is based upon estimated glomerular filtration rate (eGFR) changes over the period of time.

29. The method of claim 28, wherein the eGFR is estimated using at least one the Chronic Kidney Disease Epidemiology Collaboration (CKD-EPI) creatinine equation, the Modification of Diet in Renal Disease (MDRD) Study equation, or the Cystatin C (CysC) equation.

30. The method of claim 28, wherein progressive decline in kidney function includes an eGFR decline of ≥5 ml/min/1.73 m2/year or ≥40% sustained decline in eGFR or kidney failure (sustained eGFR <15 ml/min/1.73 m2/year.

31. The method of claim 1, wherein the risk value is a number in a range between 5 and 100 with increments of 5.

32. The method of claim 1, wherein the risk value is a number in a range between 0 and 100 with increments of 1.

33. The method of claim 1, wherein the risk value indicates a likelihood of progressive decline in kidney function.

34. The method of any of claims 1-33, further comprising:
sending, when the risk score is greater than a predetermined threshold, a signal having instruction for administering a therapy to the diabetic human subject.

35. The method of any of claims 1-34, further comprising:
detecting the risk value generated by the machine learning model is within a preset range; and
monitoring, after receiving the biomarker data and the first set of HSD or the second set of HSD, the human subject for an improvement in a level of the risk score or at least one biomarker from the biomarker data or at least one factor of the first set of HSD or the second set of HSD of the diabetic human subject.

36. The method of any of claims 1-35, further comprising:
estimating a cardiovascular disease risk based the risk value that the diabetic human subject will experience progressive decline in kidney function over the period of time.

37. The method of any of claims 1-36, further comprising:
detecting the risk value is above a preset threshold; and
sending an alarm to a compute device associated with the diabetic human subject to visit a healthcare provider.

38. The method of any of claims 1-37, further comprising:
classifying the diabetic human subject as a low risk patient, an intermediate risk patient, or a high-risk patient.

39. The method of any of claims 1-38, further comprising:
administering a therapy to reduce the risk value that the diabetic human subject will experience progressive decline in kidney function over the period of time; and
assessing a treatment effect of the therapy by calculating a trend of risk values generated over time.

40. The method of any of claims 1-38, wherein the risk value is a first risk value and the period of time is a first period of time, the method further comprising:
determining a second risk value over a second period of time, a difference between the first risk value at the first period of time and the second risk value at the second period of time being informative about a trend of risk of progressive decline in kidney function in the diabetic human subject.

41. The method of claim 40, further comprising:
training, after executing, the machine learning model based on at least one the first risk value or the second risk value.

42. The method of any of claims 1-38, wherein the risk value is a first risk value and the period of time is a first period of time, the method further comprising:
administering a therapy on the diabetic human subject; and
determining a second risk value over a second period of time, a difference between the first risk value at the first period of time and the second risk value at the second period of time being informative about the therapy administered on the diabetic human subject.

43. The method of claim 42, wherein the therapy includes at least one of a therapy based on SGLT2i (e.g. canagliflozin), angiotensin converting enzyme (ACE) inhibitors, or angiotensin-receptor blockers (ARBs).

44. The method of claim 42, wherein the therapy includes at least one of a change in lifestyle, a change in diet, or a change in exercise.

45. The method of any of claims 1-38, further comprising:
administering a first therapy in response to the risk value that the diabetic human subject will experience progressive decline in kidney function being above a pre-set threshold.
administering a second therapy in response to the risk value that the diabetic human subject will experience progressive decline in kidney function being below the pre-set threshold.

46. A non-transitory processor-readable medium storing code representing instructions to be executed by a processor of a first compute device, the code comprising code to cause the processor to:
(a) receive, from a second compute device remote from the first compute device, a trained machine learning model;
(b) receive biomarker data and a first set of HSD for a diabetic human subject, the biomarker data indicating a level of at least one of the following biomarkers: sTNFR-1, sTNFR-2, KIM-1, and ratios to one another of any of the preceding, and the first set of HSD including a metabolic factor, a health-related factor, or a demographic-related factor; and
(c) execute the trained machine learning model to generate an indication of whether the diabetic human subject will experience a progressive decline in kidney function over a period of time.

47. The non-transitory processor-readable medium of claim 46, wherein the second compute device is configured to:
receive, for each respective human training subject in a plurality of diabetic human training subjects:
a respective set of biomarker data from a biological sample collected from the respective human training subject at a respective first time;
a respective first set of human subject data (HSD) collected from the respective human training subject at the respective first time; and
a respective second set of HSD collected from the respective human training subject at a respective second time after the respective first time;
determine, for each respective human training subject in the plurality of diabetic human training subjects, a respective indication of whether the respective human training subject experienced progressive decline in kidney function based on at least the respective second set of HSD; and
train a machine learning model against, for each respective human training subject in the plurality of diabetic human training subjects (a) a plurality of features derived from at least the respective set of biomarker data collected at the respective first time and the respective first set HSD collected at the respective first time, and (b) a respective indication of whether the respective human training subject experienced progressive decline in kidney function, to produce the trained machine learning model.

48. The non-transitory processor-readable medium of claim of claim 47, wherein the trained machine learning model determines a relationship between (i) a plurality of features derived from at least the set of biomarker data and the first set of HSD and (ii) an indication of whether a diabetic human will experience progressive decline in kidney function over a period of time.

49. The non-transitory processor-readable medium of claim of claim 47, further comprising code to:
receive, for each respective human training subject in the plurality of diabetic human training subjects, a respective third set of HSD collected from the respective human training subject at a third time before the first time.

50. The non-transitory processor-readable medium of claim of claim 47, further comprising code to:
receive, for each respective human training subject in the plurality of diabetic human training subjects, a respective fourth set of HSD collected from the respective human training subject at a third time after the second time.

51. The non-transitory processor-readable medium of claim 46, wherein the biomarker data indicates a level of at least one of the following biomarkers: sTNFR-1, sTNFR-2, KIM-1, and ratios to one another of any of the preceding.

52. The non-transitory processor-readable medium of claim 51, further comprising code to cause the processor to:
detect the biomarker data of the diabetic human subject in a biological sample of the diabetic human subject.

53. The non-transitory processor-readable medium of any of claims 47, further comprising code to cause the processor to:
obtain first set of HSD or the second set of HSD of the diabetic human subject, the first set of HSD or the second set of HSD including a metabolic factor, a health-related factor, or a demographic-related factor.

54. The non-transitory processor-readable medium of claim 53, wherein the metabolic factor includes at least one of a Serum Albumin level, a Serum Calcium level, liver enzymes (AST) level, a Platelet Count, or a Glomerular Filtration Rate.

55. The non-transitory processor-readable medium of claim 53, wherein the metabolic factor includes at least one of a Hemoglobin-AlC (HbAlC) level, a Urine Albumin-Creatinine Ratio (UACR), a low density lipoprotein cholesterol level, a high density lipoprotein cholesterol level, a triglyceride level, a systolic blood pressure value, or a diastolic blood pressure value.

56. The non-transitory processor-readable medium of claim 53, wherein the health-related factor includes a body-mass-index (BMI) value, a status of past smoking, or a status of current smoking.

57. The non-transitory processor-readable medium of claim 53, wherein the demographic-related factor includes age, gender, ethnicity, income, education, or employment history.

58. The non-transitory processor-readable medium of claim 46, wherein the period of time less than 5 years.

59. The non-transitory processor-readable medium of claim 47, wherein the biomarker data and the first set of HSD of the plurality of diabetic human subjects are split into derivation data and validation data.

60. The non-transitory processor-readable medium of any of claims 56-59, wherein the plurality of diabetic human subjects are from a first population from a first geographical location and a second population from a second geographical location.

61. The non-transitory processor-readable medium of any of claims 46-59, wherein the plurality of diabetic human subjects are from a first population from a first healthcare setting and a second population from a second healthcare setting.

62. The non-transitory processor-readable medium of claim 59, wherein the derivation data are split into training data and test data.

63. The non-transitory processor-readable medium of claim 59, further comprising code to cause the processor to:
execute, after the training, the trained machine learning model based on the validation data.

64. The non-transitory processor-readable medium of any of claims 46-63, wherein the trained machine learning model includes a random forest model, deep learning model, a least absolute shrinkage and selection operator (LASSO) model, an eXtreme Gradient Boosting (XGBoost) model, or a support vector machine (SVM).

65. The non-transitory processor-readable medium of claim 47, further comprising code to cause the processor to:
performing, before training the machine learning model, a multi-fold cross-validation.

66. The non-transitory processor-readable medium of claim 47, further comprising:
classifying, before training the machine learning model, the first set of HSD of the plurality of diabetic human subjects into a plurality of non-overlapping categories.

67. The non-transitory processor-readable medium of claim 47, wherein the first set of HSD or the second set of HSD of the plurality of diabetic human samples include Related Health Problems (ICD) codes or Current Procedures Terminology (CPT) codes, each ICD code from the ICD codes or each CPT code from the CPT codes are associated with a Boolean variable and a timestamp.

68. The non-transitory processor-readable medium of any of claims 47, wherein the first set of HSD or the second set of HSD of the plurality of diabetic human subjects include medication data and laboratory values for each diabetic human subject from the plurality of diabetic human subjects.

69. The non-transitory processor-readable medium of claim 68, further comprising code to cause the processor to:
mapping, before training the machine learning model, the medication data to RxNorm codes, the training the machine learning model including training the machine learning model based on the RxNorm codes.

70. The non-transitory processor-readable medium of claim 68, further comprising code to cause the processor to:
mapping, before training the machine learning model, the laboratory values to Logical Observation Identifiers Names and Codes (LOINC) code, the training the machine learning model including training the machine learning model based on the LOINC codes.

71. The non-transitory processor-readable medium of claims 46, wherein the trained machine learning model is configured to predict a composite kidney endpoint of progressive decline in kidney function.

72. The non-transitory processor-readable medium of claim 46, wherein progressive decline in kidney function is based upon an estimated glomerular filtration rate (eGFR) changes over the period of time.

73. The non-transitory processor-readable medium of claim 72, wherein the eGFR is estimated using at least one of the Chronic Kidney Disease Epidemiology Collaboration (CKD-EPI) creatinine equation, the Modification of Diet in Renal Disease (MDRD) Study equation, or the Cystatin C (CysC) equation.

74. The non-transitory processor-readable medium of claim 72, wherein progressive decline in kidney function includes an eGFR decline of ≥5 ml/min/1.73 m2/year or ≥40% sustained decline in eGFR or kidney failure (sustained eGFR <15 ml/min/1.73 m2/year.

75. The non-transitory processor-readable medium of claim 46, wherein the risk value is a number in a range between 5 and 100 with increments of 5.

76. The non-transitory processor-readable medium of claim 46, wherein the risk value is a number in a range between 0 and 100 with increments of 1.

77. The non-transitory processor-readable medium of claim 46, wherein the risk value indicates a likelihood of progressive decline in kidney function.

78. The non-transitory processor-readable medium of any of claims 46-77, further comprising code to cause the processor to:
send, when the risk score is greater than a predetermined threshold, a signal having instruction for administering a therapy to the diabetic human subject.

79. The non-transitory processor-readable medium of any of claims 46-78, further comprising code to cause the processor to:
detect the risk value generated by the trained machine learning model is within a preset range; and
monitor, after receiving, the biomarker data and the first set of HSD, the human subject for an improvement in a level of the risk score or at least one biomarker from the biomarker data or at least one factor of the first set of HSD data of the diabetic human subject.

80. The non-transitory processor-readable medium of any of claims 46-79, further comprising code to cause the processor to:
estimate a cardiovascular disease risk based the risk value that the diabetic human subject will experience progressive decline in kidney function over the period of time.

81. The non-transitory processor-readable medium of any of claims 46-80, further comprising code to cause the processor to:
detect the risk value is above a preset threshold; and
send an alarm to a compute device associated with the diabetic human subject to visit a healthcare provider.

82. The non-transitory processor-readable medium of any of claims 46-81, further comprising code to cause the processor to:
classify the diabetic human subject as a low risk patient, an intermediate risk patient, or a high-risk patient.

83. The non-transitory processor-readable medium of any of claims 46-82, further comprising code to cause the processor to:
administer a therapy to reduce the risk value that the diabetic human subject will experience progressive decline in kidney function over the period of time; and
assess a treatment effect of the therapy by calculating a trend of risk values generated over time.

84. The non-transitory processor-readable medium of any of claims 46-82, wherein the risk value is a first risk value and the period of time is a first period of time, the medium further comprising code to cause the processor to:
determine a second risk value over a second period of time; a difference between the first risk value at the first period of time and the second risk value at the second period of time being informative about a change in the diabetic human subject.

85. The non-transitory processor-readable medium of claim 84, further comprising code to cause the processor to:
train, after executing, the trained machine learning model based on at least one the first risk value or the second risk value.

86. The non-transitory processor-readable medium of any of claims 46-82, wherein the risk value is a first risk value and the period of time is a first period of time, the medium further comprising code to cause the processor to:
administer a therapy on the diabetic human subject; and
determine a second risk value over a second period of time; a difference between the first risk value at the first period of time and the second risk value at the second period of time being informative about the therapy administered on the diabetic human subject.

87. The non-transitory processor-readable medium of claim 86, wherein the therapy includes at least one of a therapy based on SGLT2i canagliflozin, angiotensin converting enzyme (ACE) inhibitors, or angiotensin-receptor blockers (ARBs).

88. The non-transitory processor-readable medium of claim 86, wherein the therapy includes at least one of a change in lifestyle, a change in diet, or a change in exercise.

89. The non-transitory processor-readable medium of any of claims 46-82, further comprising code to cause the processor to:
administer a first therapy in response to the risk value that the diabetic human subject will experience progressive decline in kidney function being above a pre-set threshold.
administer a second therapy in response to the risk value that the diabetic human subject will experience progressive decline in kidney function being below the pre-set threshold.

90. A method, comprising:
detecting biomarker data collected from a plurality of biological samples from a plurality of diabetic human subjects, each biomarker datum from the biomarker data indicating a level of at least one of the following biomarkers: sTNFR-1, sTNFR-2, KIM-1, and ratios to one another of any of the preceding;
obtaining a first set of human subject data (HSD) of the plurality of diabetic human subjects at a first time,
obtaining a second set of human subject data (HSD) of the plurality of diabetic human subjects at a second time, the first set of HSD or the second set of HSD each including a metabolic factor, a health-related factor, or a demographic-related factor; and
determining, for each diabetic human subject in the plurality of diabetic human subjects, an indication of whether the diabetic human subject experienced progressive decline in kidney function based on at least the second set of HSD.

91. The method of claim 90, wherein the metabolic factor includes at least one of a Serum Albumin level, a Serum Calcium level, liver enzymes (AST) level, a Platelet Count, or a Glomerular Filtration Rate.

92. The method of claim 90, wherein the metabolic factor includes at least one of a Hemoglobin-AlC (HbAlC) level, a Urine Albumin-Creatinine Ratio (UACR), a low density lipoprotein cholesterol level, a high density lipoprotein cholesterol level, a triglyceride level, a systolic blood pressure value, or a diastolic blood pressure value.

93. The method of claim 90, wherein the health-related factor includes at least one of a body-mass-index (BMI) value, a status of past smoking, or a status of current smoking.

94. The method of claim 90, wherein the demographic-related factor includes at least one of an age, a gender, an ethnicity, an income, an education, or an employment history.

95. The method of claim 90, wherein the period of time less than 5 years.

96. The method of claim 90, further comprising:
training a machine learning model against, for each diabetic human subject in the plurality of diabetic human subjects (a) a plurality of features derived from at least the set of biomarker data collected at the first time and the first set HSD collected at the first time, and (b) a indication of whether the diabetic human subject experienced progressive decline in kidney function;
receiving a set of biomarker data and a first set of HSD, for a human test subject not included in the plurality of diabetic human subjects, collected at a first time for the human test subject; and
executing, after the training, the machine learning model to generate an indication of whether the human test subject will experience progressive decline in kidney function over the period of time, based on the set of biomarker data and the first set of HSD for the human test subject.

97. The method of claim 96, wherein the machine learning model determines a relationship between (i) a plurality of features derived from at least the set of biomarker data and the first set of HSD and (ii) an indication of whether a diabetic human will experience progressive decline in kidney function over a period of time.

98. The method of claim 96, further comprising:
receiving, for each respective human training subject in the plurality of diabetic human training subjects, a respective third set of HSD collected from the respective human training subject at a third time before the first time.

99. The method of claim 96, further comprising:
receiving, for each respective human training subject in the plurality of diabetic human training subjects, a respective fourth set of HSD collected from the respective human training subject at a third time after the second time.

100. The method of claim 96, wherein the biomarker data and the first set of HSD of the plurality of diabetic human subjects are split into derivation data and validation data.

101. The method of any of claims 90-100, wherein the plurality of diabetic human subjects are from a first population from a first geographical location and a second population from a second population.

102. The method of any of claims 90-100, wherein the plurality of diabetic human subjects are from a first population from a first healthcare setting and a second population from a second healthcare setting.

103. The method of claim 96, wherein the derivation data are split into training data and test data.

104. The method of claim 96, further comprising:
executing, after the training, the machine learning model based on the validation data.

105. The method of claim 96, wherein the machine learning model includes a random forest model, deep learning model, a least absolute shrinkage and selection operator (LASSO) model, an eXtreme Gradient Boosting (XGBoost) model, or a support vector machine (SVM).

106. The method of claim 96, further comprising:
performing, before training the machine learning model, a multi-fold cross-validation.

107. The method of claim 96, further comprising:
classifying, before training the machine learning model, the first set of HSD of the plurality of diabetic human subjects into a plurality of non-overlapping categories.

108. The method of any of claims 90, wherein the first set of HSD or the second set of HSD of the plurality of diabetic human samples include Related Health Problems (ICD) codes or Current Procedures Terminology (CPT) codes, each ICD code from the ICD codes or each CPT code from the CPT codes are associated with a Boolean variable and a timestamp.

109. The method of claim 96, wherein the first set of HSD or the second set HSD of the plurality of diabetic human subjects include medication data and laboratory values for each diabetic human subject from the plurality of diabetic human subjects.

110. The method of claim 91, further comprising:
mapping, before training the machine learning model, the medication data to RxNorm codes, the training the machine learning model including training the machine learning model based on the RxNorm codes.

111. The method of claim 91, further comprising:
mapping, before training the machine learning model, the laboratory values to Logical Observation Identifiers Names and Codes (LOINC) code, the training the machine learning model including training the machine learning model based on the LOINC codes.

112. The method of claim 96, wherein the machine learning model is configured to predict a composite kidney endpoint of progressive decline in kidney function.

113. The method of claim 90, wherein progressive decline in kidney function is based upon an estimated glomerular filtration rate (eGFR) changes over the period of time.

114. The method of claim 113, wherein the eGFR is estimated using at least one of the Chronic Kidney Disease Epidemiology Collaboration (CKD-EPI) creatinine equation, the Modification of Diet in Renal Disease (MDRD) Study equation, or the Cystatin C (CysC equation.

115. The method of claim 113, wherein progressive decline in kidney function includes an eGFR decline of ≥5 ml/min/1.73 m2/year or ≥40% sustained decline in eGFR or kidney failure (sustained eGFR <15 ml/min/1.73 m2/year.

116. The method of claim 90, wherein the risk value is a number in a range between 5 and 100 with increments of 5.

117. The method of claim 90, wherein the risk value is a number in a range between 0 and 100 with increments of 1.

118. The method of claim 90, wherein the risk value indicates a likelihood of progressive decline in kidney function.

119. The method of any of claims 90-118, further comprising:
sending, when the risk score is greater than a predetermined threshold, a signal having instruction for administering a therapy to the diabetic human subject.

120. The method of any of claim 96, further comprising:
detecting the risk value generated by the machine learning model is within a preset range; and
monitoring, after receiving, the biomarker data and the first set of HSD, the human subject for an improvement in a level of the risk score or at least one biomarker from the biomarker data or at least one factor of the first set of HSD of the diabetic human subject.

121. The method of any of claims 90-120, further comprising:
estimating a cardiovascular disease risk based the risk value that the diabetic human subject will experience progressive decline in kidney function over the period of time.

122. The method of any of claims 90-121, further comprising:
detecting the risk value is above a preset threshold; and
sending an alarm to a compute device associated with the diabetic human subject to visit a healthcare provider.

123. The method of any of claims 90-122, further comprising:
classifying the diabetic human subject as a low risk patient, an intermediate risk patient, or a high-risk patient.

124. The method of any of claims 90-123, further comprising:
administering a therapy to reduce the risk value that the diabetic human subject will experience progressive decline in kidney function over the period of time; and
assessing a treatment effect of the therapy by calculating a trend of risk values generated over time.

125. The method of any of claims 90-124, wherein the risk value is a first risk value and the period of time is a first period of time, the medium further comprising code to cause the processor to:
determining a second risk value over a second period of time; a difference between the first risk value at the first period of time and the second risk value at the second period of time being informative about a change in the diabetic human subject.

126. The method of any of claims 90-125, wherein the risk value is a first risk value and the period of time is a first period of time, the medium further comprising code to cause the processor to:
administering a therapy on the diabetic human subject; and
determining a second risk value over a second period of time; a difference between the first risk value at the first period of time and the second risk value at the second period of time being informative about the therapy administered on the diabetic human subject.

127. The method of claim 126, wherein the therapy includes at least one of a therapy based on SGLT2i canagliflozin, angiotensin converting enzyme (ACE) inhibitors, or angiotensin-receptor blockers (ARBs).

128. The method of claim 126, wherein the therapy includes at least one of a change in lifestyle, a change in diet, or a change in exercise.

129. The method of any of claims 90-124, further comprising:
administering a first therapy in response to the risk value that the diabetic human subject will experience progressive decline in kidney function being above a pre-set threshold.
administering a second therapy in response to the risk value that the diabetic human subject will experience progressive decline in kidney function being below the pre-set threshold.
